(19) United States
(12) Patent Application Publication
(10) Pub. No.: US 2023/0210736 A1
(43) Pub. Date: Jul. 6, 2023

(54) HAIR TREATMENT COMPOSITION

(71) Applicant: L'OREAL, Paris (FR)

(72) Inventors: Kazumitsu KAWAKAMI, Westfield, NJ (US); Ronak Rughani, Edison, NJ (US); Liliana Xavier, Elizabeth, NJ (US); Aakash Parekh, Edison, NJ (US)

(21) Appl. No.: 18/146,193

(22) Filed: Dec. 23, 2022

Related U.S. Application Data
(60) Provisional application No. 63/294,897, filed on Dec. 30, 2021.

(30) Foreign Application Priority Data
Jan. 21, 2022 (FR) 2201499

(51) Int. Cl. A61K 8/365; A61K 8/06; A61Q 5/08; A61Q 5/02; A61K 8/34; A61K 8/41; A61K 8/44; A61K 8/73; A61K 8/891; A61K 8/898

(52) U.S. Cl. CPC A61K 8/365; A61K 8/062; A61Q 5/08; A61Q 5/02; A61K 8/342; A61K 8/416; A61K 8/44; A61K 8/737; A61K 8/891; A61K 8/898; A61K 2800/48; A61K 2800/5422

(57) ABSTRACT

Hair treatment compositions and methods of using them to treat hair are described. The hair treatment compositions include: (a) citric acid, one or more salts of citric acid, or a combination thereof; or a combination of citric acid and one or more salts of citric acid, wherein the weight ratio of citric acid to salts of citric acid added to form the combination is from 1:10 to 10:1; (b) one or more surfactants; and (c) water; wherein the pH of the composition is from about 3 to about 10; and weight percentages are based on the total weight of the composition. The hair treatment compositions are particularly useful for strengthening hair fibers.

### CROSS-REFERENCE TO RELATED APPLICATIONS

This application claims benefit of U.S. Ser. No. 63/294,897 filed Dec. 30, 2021, and benefit of French Application No. FR 2201499, filed on Jan. 21, 2022, which are incorporated herein by reference in their entirety.

### FIELD OF THE DISCLOSURE

The present disclosure relates to hair-treatment compositions and methods for treating hair with the hair treatment compositions.

### BACKGROUND

Many consumers desire to use cosmetic and care compositions that enhance the appearance of keratinous substrates such as hair, e.g., by changing the color, style, and/or shape of the hair, and/or by imparting various cosmetic properties to hair, such as shine and conditioning. Many of the known compositions and processes for enhancing the appearance of hair involve chemical treatments to the hair.

The process of changing the color of hair, for example, can involve depositing an artificial color onto the hair which provides a different shade or color to the hair, and/or lifting the color of the hair, such as lightening the color of dark hair to lighter shades. The process of lifting the color of hair, also known as lightening (or bleaching), generally requires the use of oxidizing agents. Lightening of hair is typically evaluated by the variation in tone height before and after the application of a hair color-altering composition onto hair. This variation corresponds to the degree or level of lightening or lift. The notion of “tone” is based on the classification of the natural shades, one tone separating each shade from the shade immediately following or preceding it, which is well known to hairstyling professionals. The tone heights or levels can range from 1 (black) to 10 (light blond), one unit corresponding to one tone; thus, the higher the number, the lighter the shade or the greater the degree of lift.

In general, hair lightening or color lifting compositions and hair coloring or dyeing compositions possess an alkalinity such that these compositions have a pH value of above 7, typically being at pH 9 and above, and may generally require the presence of an alkalizing agent such as ammonia or an ammonia gas generating compound and/or an amine or ammonium-based compound in amounts sufficient to make such compositions alkaline. The alkalizing agent causes the hair shaft to swell, thus allowing the small oxidative dye molecules to penetrate the cuticle and cortex before the oxidation condensation process is completed. The resulting larger-sized colored complexes from the oxidative reaction are then trapped inside the hair fiber, thereby permanently altering the color of the hair.

Additionally, there are many techniques and compositions for styling or altering the shape of hair. For example, hair care products referred to as “hair relaxers” or “hair straighteners” can relax or straighten curly or kinky hair, including wavy hair. Straightening or relaxing the curls of very curly hair may increase the manageability and ease of styling of such hair. Compositions for permanent waving the hair will impart a curl or a wave to otherwise straight hair. Different types of compositions can be applied onto hair in order to change its shape and make it more manageable, such as alkaline and acidic compositions. Hair relaxers, straighteners, perms, and/or waves may either be applied in a hair salon by a professional or in the home by the individual consumer.

While dyeing or color lifting compositions can effectively alter the color of hair, and relaxing, straightening, perming, and waving compositions can effective alter the shape of the hair, these chemical treatments can damage the hair fibers and/or irritate the scalp. Thus, in order to reduce or avoid damage to hair, as well as to improve the cosmetic performance of the compositions, the use of new and additional components and novel combinations of ingredients are continuously sought.

However, the choice of components or combinations of ingredients could pose difficulties insofar as they cannot be detrimental to other cosmetic attributes such as ease and uniformity of application, rheology or viscosity properties and stability of the compositions, color deposit and target shade formation, and/or result into more disadvantages such as increased damage or a less healthy look to the hair. It would therefore be desirable to provide the consumer with compositions and methods that can chemically treat the hair while providing other cosmetic advantages such as shine, conditioning, fiber strength, and/or a healthy appearance to the hair, but avoiding or minimizing damage to the hair.

Further, both natural and sensitized or chemically treated hair can contain several kinds of negatively charged moieties, for example, carboxylates (resulting from the hydrolysis of amino acids and thioester bonds) and/or sulfonates (resulting from the oxidation of disulfide bonds). These negatively charged moieties can degrade the cosmetic properties of the hair. Moreover, when hair is chemically treated or damaged, the disulfide bonds in hair (disulfide linkages between two cysteine units) can be reduced or broken, resulting in the formation of thiol groups and/or cysteic acid.

Thus, one objective of the disclosure is to provide novel compositions that can provide advantageous effects such as strengthening of the hair fiber, protecting hair fibers from damage or further damage, enhanced properties such as softness, shine, conditioning, healthy appearance, while at the same time, providing desired cosmetic.

### SUMMARY OF THE DISCLOSURE

The instant disclosure relates to hair treatment compositions and methods for treating hair, for example, the hair of the head. The compositions and methods dramatically improve the quality and durability of have, especially chemically treated hair. Damage caused to hair during or resulting from a chemical treatments is repaired, minimized, and/or compensated for due to a unique combination of components in the compositions that interact to restructure, strengthen, and/or protect the keratin fibers of the hair. The compositions also prevent, mitigate, and/or treat damage to the hair caused by environmental factors (e.g., hard water, sun damage, chlorine, etc.) and styling the hair, for example, with heat (e.g., blow drying, hot irons, etc.). Hair treated with the compositions is surprisingly strengthened and the hair's cosmetic properties (e.g., softness, smoothness, and discipline) are considerably improved.

In an embodiment, the hair treatment compositions include: (a) about 0.25 to about 10 wt. % of citric acid, one or more salts of citric acid, or a combination thereof, wherein if a combination of citric acid and salts of citric acid is present, the weight ratio of citric acid to salts of citric acid added to form the combination is from 1:10 to about 10:1; or the hair treatment composition comprises about 1 to about 10 wt. % of a combination of citric acid and one or more salts of citric acid, wherein the weight ratio of citric acid to salts of citric acid added to the hair treatment composition to form the combination is from 1:10 to 10:1; (b) one or more surfactants; and (c) water; wherein the pH of the composition is from about 3 to about 10; and weight percentages are based on the total weight of the composition.

Nonlimiting examples of salts of citric acid include sodium citrate, sodium citrate tribasic, citric acid trisodium salt, trisodium citrate, sodium citrate dihydrate, sodium citrate tribasic dihydrate, citric acid trisodium salt dihydrate, trisodium citrate dihydrate, potassium citrate, tripotassium citrate, potassium citrate tribasic, citric acid tripotassium salt, potassium citrate monohydrate, tripotassium citrate monohydrate, potassium citrate tribasic monohydrate, citric acid tripotassium salt monohydrate, citric acid disodium salt, citric acid disodium salt sesquihydrate, sodium hydrogencitrate, sodium hydrogencitrate sesquihydrate, disodium hydrogen citrate, disodium hydrogen citrate sesquihydrate, sodium citrate dibasic, sodium citrate dibasic sesquihydrate, potassium citrate monobasic, potassium dihydrogen citrate, citric acid monopotassium salt, sodium citrate monobasic, sodium dihydrogen citrate, citric acid monosodium salt, and the like. In a preferred embodiment, the salts of citric acid include sodium citrate (monosodium citrate, disodium citrate, and/or trisodium citrate), potassium citrate (also known as tripotassium citrate), and combinations thereof.

In various embodiments, the one or more surfactants of (b) include or consist of one or more cationic surfactants. Nonlimiting examples of useful cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof.

In various embodiments, at least one of the one or more surfactants of (b) is a nonionic surfactant, an amphoteric surfactant, or a combination thereof.

In various embodiments, the hair treatment compositions are in the form of an emulsion, for example, an oil-in-water emulsion or a water-in-oil emulsion, preferably an oil-in-water emulsion. Fatty alcohols and additional fatty compounds (also referred to as “fatty materials”) form at least part of the fatty phase (oil phase) of the emulsions. Accordingly, in various embodiments, the hair treatment compositions include: (d) one or more fatty alcohols; and (e) one or more fatty compounds other than fatty alcohols.

The aqueous phase includes water, and optionally water-soluble components, for example, one or more water-soluble solvents. The compositions typically include high amounts of water, for example, at least 50% by weight, preferably at least 55% by weight, and more preferably, at least 60% by weight.

Fatty alcohols are well known in the art, and include aliphatic alcohols, for example, primary alcohols having a carbon chain from about 8 to about 30 carbon atoms, preferably from about 12 to about 22 carbon atoms. Non-limiting examples of fatty alcohols include decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol, and a mixture thereof. In some cases, the fatty alcohols comprise at least one of, or may be chosen from, myristyl alcohol, lauryl alcohol, cetyl alcohol, stearyl alcohol, cetearyl alcohol, isostearyl alcohol, oleyl alcohol, isotridecyl alcohol, and a mixture thereof.

Fatty compounds other than the fatty alcohols discussed above may optionally be included in the hair treatment compositions. The term “fatty compound” is interchangeable with the term “fatty material.” As is well known in the art, fatty compounds are compounds that are not soluble (or only sparingly soluble in water; they are hydrophilic and can often be solubilized in organic solvents). They include materials such as oils, fats, waxes, hydrocarbons, fatty esters, fatty acids, etc. Non-limiting examples of useful fatty compounds include oils, waxes, alkanes (paraffins), fatty acids, fatty esters, triglyceride compounds, lanolin, hydrocarbons, derivatives thereof, and mixtures thereof.

In various embodiments, the hair treatment compositions include both: (d) one or more fatty alcohols; and (e) one or more fatty compounds other than fatty alcohols. The weight ratio of the (d) one or more fatty alcohols to the (e) one or more fatty compounds other than fatty alcohols can vary. Nonetheless, in certain embodiments the weight ratio is from about 2:1 to about 15:1, preferably from about 3:1 to about 10:1, more preferably from about 4:1 to about 8:1.

In various embodiments, the hair treatment compositions include: (f) one or more water-soluble solvents. Nonlimiting examples of useful water-soluble solvents include glycerin, C₁₋₆ mono-alcohols, polyols (polyhydric alcohols), glycols, and a mixture thereof. In certain embodiments, it is preferrable for the hair treatment composition to include at least glycerin, one or more C₁₋₆ mono-alcohols, or a mixture thereof.

In various embodiments, the hair treatment composition includes: (g) one or more nonionic thickening polymers. Nonlimiting examples of useful nonionic thickening polymers include guar gum, guar derivatives, cellulose gum, cellulose derivatives, starch, starch derivatives, polysaccharides, polysaccharide derivatives, homopolymers and copolymers of ethylene oxide having a molar mass equal to or greater than 10,000 g/mol, polyvinyl alcohols, homopolymers and copolymers of vinylpyrrolidone, homopolymers and copolymers of vinylcaprolactam, homopolymers and copolymers of polyvinyl methyl ether, and mixtures thereof. In certain embodiments, guar gum and/or guar derivatives (for example hydroxypropyl guar) are particularly useful.

In various embodiments, the hair treatment composition include: (h) one or more conditioning agents. Nonlimiting examples of conditioning agents include cationic polymers and silicones. In certain embodiments, the hair treatment compositions include one or more cationic polymers. Nonlimiting examples of cationic polymers include cationic cellulose derivatives, quaternized hydroxyethyl cellulose (e.g., polyquaternium-4, polyquaternium-10, polyquaternium-24, etc.), cationic starch derivatives, cationic guar gum derivatives, cationic proteins and cationic protein hydrolysates, quaternary diammonium polymers, copolymers of acrylamide and dimethyldiallyammonium chloride, polyquaterniums (e.g., polyquaternium-10), and a mixture thereof.

In certain embodiments the hair treatment compositions include one or more silicones. Nonlimiting examples of silicones include dimethicone, dimethiconol, cyclomethicone, polysilicone-11, phenyl trimethicone, amodimethicone, bis-cetearyl amodimethicone, and a mixture thereof.

The hair treatment compositions of the instant disclosure, in various embodiments, are part of a kit, for example, a kit comprising one or more hair treatment compositions according to the instant disclosure and one or more additional hair treatment compositions, wherein each of the one or more hair treatment compositions (and each of the additional hair treatment compositions) are separately contained. For instance, in certain embodiments, the kits comprise: (i) one or more hair treatment compositions according to the instant disclosure; and (ii) one or more additional hair treatment compositions chosen from hair cleansing compositions (shampoos), hair conditioning compositions (conditioners), and hair treatment compositions for chemically treating hair comprising one or more chemically active agents that chemically treat the hair.

The hair treatment compositions are particularly useful in methods for treating hair, for example methods for restructuring, repairing, strengthening, and/or protecting hair. The methods repair, minimize, mitigate, and/or compensate for damage caused to hair by chemical treatments (e.g., bleaching, coloring, straightening, curling, etc.). The methods also relate to preventing, minimizing, mitigating, and treating hair vulnerable to damage (or already damaged) by environmental factors (e.g., hard water, sun damage, chlorine, etc.) and to damage caused by styling the hair, for example, with heat (e.g., blow drying, hot irons, etc.).

The methods include applying one or more hair treatment compositions of the instant disclosure to the hair. The hair treatment composition(s) may be temporarily applied to the hair (e.g., applied for a period of about 1 hour or less and rinsed from the hair), or may be applied to the hair and allowed to remain on the hair for a longer period of time before being rinsed from the hair (e.g., for about 2 minutes, 5 minutes, 10 minutes, 20 minutes, 30 minutes, or longer). In situations where the hair treatment composition is rinsed from the hair shortly after application (immediately after application or within about 2, 5, 10, 20, 30 minutes or longer) before styling the hair, the hair treatment compositions is a “rinse off” product. In certain embodiments, the hair treatment compositions are applied to the hair and allowed to remain on the hair indefinitely, for example, allowed to remain on the hair without rinsing from the hair before styling the hair, i.e., the hair treatment composition is a “leave-on” product. The methods are particularly well suited for application to chemically treated hair, hair to be subjected to a chemical treatment, damaged hair, and natural hair, for example, to improve the hair's strength, luster, shine, and/or elasticity, etc.

The hair treatment compositions of the instant case are particularly advantageous because they can be a stand-alone product (used independently) or can be used in methods that combine the hair treatment compositions of the instant disclosure with an independent hair treatment composition for chemically treating hair (i.e., a hair treatment composition comprising one or more chemically active agents that chemically treat the hair). Nonlimiting examples of hair treatment compositions for chemically treating hair include bleaching compositions, hair straightening or curling compositions, hair relaxing or texlaxing compositions, and the like. The hair treatment compositions of the instant case may be combined with (added into) one or more components of a hair treatment composition for chemically treating hair. For example, if the hair treatment composition for chemically treating hair is a bleaching composition, the hair treatment compositions of the instant case may be added into a developer component, may be added into a bleaching component, or may be combined with a mixture of the developer component and the bleaching component. Similarly, hair shaping compositions (for perming, straightening, and/or relaxing hair) often require more than one component (e.g., a relaxing component, an activator component, a neutralizer component, etc.). The hair treatment compositions of the instant disclosure may be included in one or more (or all) of the components.

Similar to the above, the hair treatment compositions of the instant disclosure may be used as a stand alone product in a hair cleansing (shampooing) and optional conditioning routine, or may be combined with (added into) a hair cleanser (shampoo), conditioner, or added into both a hair cleanser and conditioner.

Other features and iterations of the instant disclosure and its various inventions are described in more detail below.

### BRIEF DESCRIPTION OF THE DRAWINGS

Implementation of the present technology will now be described, by way of example only, with reference to the attached figures, wherein:

FIG. 1 is a schematic stress-strain curve for dry hair identifying the Young's modulus, plateau load, break force, and break extension;

FIG. 2 shows break stress results (gmf/sq micron) for treated European medium brown hair;

FIG. 3 shows Young's modulus results for treated European medium brown hair;

FIG. 4 shows extension results (% strain) for treated European medium brown hair;

FIG. 5 shows break stress results (gmf/sq micron) for treated curly multi-ethnic brown hair;

FIG. 6 shows Young's modulus results for treated curly multi-ethnic brown hair;

FIG. 7 shows break extension results (% strain) for treated curly multi-ethnic brown hair; and

FIG. 8 shows the results obtained from Cyclic Fatigue Tensile Testing (cycles to break) for hair treated with a control versus hair treated with a composition described herein.

It should be understood that the various aspects are not limited to the arrangements and instrumentality shown in the drawings.

### DETAILED DESCRIPTION OF THE DISCLOSURE

The instant disclosure relates to hair treatment compositions and methods for treating hair, in particular human hair of the head. The hair treatment compositions and methods strengthen hair, minimize and/or mitigate damage to hair, especially chemically treated hair, and prevent or reduce hair's susceptibility to damage. Thus, the hair treatment compositions of the instant disclosure can be referred to “hair restructuring compositions.” Hair treated with the hair treatment compositions (or hair restructuring compositions) exhibits improved and desirable cosmetic properties such as smoothness, gloss, improved combability, and improved strength and elasticity.

(a) Citric Acid and Salts

Citric acid is an organic compound with the chemical formula HOC(CO₂H)(CH₂CO₂H)₂hen part of a salt, the formula of the citrate anion is written as C₆H₅O₃⁻⁷ or C₃H₅O(COO)₃⁻³. Nonlimiting examples of salts of citric acid include sodium citrate, sodium citrate tribasic, citric acid trisodium salt, trisodium citrate, sodium citrate dihydrate, sodium citrate tribasic dihydrate, citric acid trisodium salt dihydrate, trisodium citrate dihydrate, potassium citrate, tripotassium citrate, potassium citrate tribasic, citric acid tripotassium salt, potassium citrate monohydrate, tripotassium citrate monohydrate, potassium citrate tribasic monohydrate, citric acid tripotassium salt monohydrate, citric acid disodium salt, citric acid disodium salt sesquihydrate, sodium hydrogencitrate, sodium hydrogencitrate sesquihydrate, disodium hydrogen citrate, disodium hydrogen citrate sesquihydrate, sodium citrate dibasic, sodium citrate dibasic sesquihydrate, potassium citrate monobasic, potassium dihydrogen citrate, citric acid monopotassium salt, sodium citrate monobasic, sodium dihydrogen citrate, citric acid monosodium salt, and the like. In a preferred embodiment, the salts of citric acid include sodium citrate (monosodium citrate, disodium citrate, and/or trisodium citrate), potassium citrate (also known as tripotassium citrate), and combinations thereof.

The total amount of citric acid, one or more salts of citric acid, or a combination of citric acid and one or more salts of citric acid will vary. For purposes of the instant disclosure, the weight percentage for the combination of the citric acid and salts of citric acid includes the citric acid, the citrate anion, and the cation of the salt, which may be dissociated from the citrate anion. Salts of citric acid are soluble in water and therefore reversibly dissociate into electrolytes. The total amount of citric acid, one or more salts of citric acid, or a combination of citric acid and one or more salts of citric acid may be from about 0.25 to about 10 wt. %, about 0.25 to about 6 wt. %, about 0.25 to about 5 wt. %, about 0.25 to about 3 wt. %, about 0.25 to about 2 wt. %, about 0.5 to about 10 wt. %, about 0.5 to about 6 wt. %, about 0.5 to about 5 wt. %, about 0.5 to about 3 wt. %, about 0.5 to about 2 wt. %, including any combination, sub-combination, range, or sub-range thereof by weight, based on the total weight of the hair treatment composition. As already noted, when a combination of citric acid and one or more salts of citric acid are present, the weight ratio of citric acid to salts of citric acid added to form the combination is from 1:10 to about 10:1

In certain embodiments, the composition includes a combination of citric acid and one or more salts of citric acid. When a combination of citric acid and one or more salts of citric acid are present, the weight ratio of citric acid to salts of citric acid added to form the combination is from 1:10 to about 10:1 The total amount of the combination of citric acid and salts of citric acid will vary. For purposes of the instant disclosure, the weight percentage for the combination of the citric acid and salts of citric acid includes the citric acid, the citrate anion, and the cation of the salt, which may be dissociated from the citrate anion. Salts of citric acid are soluble in water and therefore reversibly dissociate into electrolytes. In various embodiments, the total amount of the combination of citric acid and salts of citric acid (citric acid, citrate anions, and cations from the salt) is from about 1 to about 10 wt. %, based on the total weight of the hair treatment compositions. In further embodiments, the total amount of the combination of citric acid and salts of citric acid is from about 1 to about 8 wt. %, about 1 to about 6 wt. %, about 1 to about 5 wt. %, about 1 to about 4 wt. %, about 1.3 to about 10 wt. %, about 1.3 to about 8 wt. %, about 1.3 to about 6 wt. %, about 1.3 to about 5 wt. %, about 1.3 to about 4 wt. %, about 1.5 to about 10 wt. %, about 1.5 to about 8 wt. %, about 1.5 to about 6 wt. %, about 1.5 to about 5 wt. %, about 1.5 to about 4 wt. %, about 1.8 to about 10 wt. %, about 1.8 to about 8 wt. %, about 1.8 to about 6 wt. %, about 1.8 to about 5 wt. %, about 1.8 to about 4 wt. %, about 1.9 to about 10 wt. %, about 1.9 to about 8 wt. %, about 1.9 to about 6 wt. %, about 1.9 to about 5 wt. %, about 1.9 to about 4 wt. %, about 2 to about 10 wt. %, about 2 to about 8 wt. %, about 2 to about 6 wt. %, about 2 to about 5 wt. %, or about 2 to about 4 wt. %, or about 1.7, 1.8, 1.9, 2, 2.1, or 2.2 wt. %, including any combination, sub-combination, range, or sub-range thereof by weight, based on the total weight of the hair treatment composition.

Ratio of Citric Acid to Salts Thereof

When a combination of citric acid and one or more salts of citric acid is present, the weight ratio of citric acid to salts of citric added into the cosmetic composition will vary. Nonetheless, in various embodiments the weight ratio of the citric acid to the salts of citric acid added into the cosmetic composition is from about 1:10 to about 10:1. If further embodiments, the weight ratio of the citric acid to the salts of citric acid added into the cosmetic composition is from about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1, about 1.8:1 to about 1:1.8, about 1.6:1 to about 1:1.6, about 1.5:1 to about 1:1.5, or about 2:1, about 1.8:1, about 1.7:1, about 1.6:1, about 1.5:1, about 1.4:1, about 1.3:1, about 1.2:1, or about 1.1:1, including any combination, sub-combination, range, or sub-range thereof. In yet further embodiments, the weight ratio of the citric acid to the salts of citric acid added into the cosmetic composition is greater than 1:1, i.e., the amount of citric acid added into the cosmetic composition is greater than the amount of salt(s) of citric acid added into the hair treatment composition. For example, in certain embodiments, the ratio is greater than 1:1 to about 10:1, about 8:1, about 6:1, about 5:1, about 4:1, about 3:1, or about 2:1.

(b) Surfactants

The hair treatment compositions of the instant disclosure include one or more surfactants, for example, one or more surfactants chosen from cationic surfactants, nonionic surfactants, amphoteric surfactants (or zwitterionic surfactants), and anionic surfactants. In various embodiments, the hair treatment compositions include at least one cationic surfactant. For example, the hair treatment compositions may include at least one cationic surfactant and optionally, one or more additional surfactants chosen from nonionic surfactants, amphoteric surfactants, and anionic surfactants. Preferably, however, when the hair treatment composition includes at least one cationic surfactant, the hair treatment composition is free or essentially free from anionic surfactants. Accordingly, in various embodiments, the hair treatment composition comprises at least one cationic surfactant and optionally, one or more additional surfactants chosen from nonionic surfactants, amphoteric surfactants, and a combination thereof.

The total amount of all surfactants (cationic, nonionic, amphoteric, and anionic surfactants) in the hair treatment composition will vary. Nonetheless, in various embodiments, the total amount of all surfactants in the hair treatment composition is from about 1 to about 15 wt. %, based on the total weight of the hair treatment composition. In further embodiments, the total amount of all surfactants in the hair treatment composition is from about 1 to about 12 wt. %, about 1 to about 10 wt. %, about 1 to about 8 wt. %, about 2 to about 15 wt. %, about 2 to about 12 wt. %, about 2 to about 10 wt. %, about 2 to about 8 wt. %, about 3 to about 15 wt. %, about 3 to about 12 wt. %, about 3 to about 10 wt. %, about 3 to about 8 wt. %, about 4 to about 15 wt. %, about 4.5 to about 12 wt. %, about 4.5 to about 10 wt. %, about 4.5 to about 8 wt. %, or about 4.6, 4.7, 4.8, 4.9, 5, 5.1 wt. %, including any combination, sub-combination, range, or sub-range thereof by weight, based on the total weight of the hair treatment composition.

(i) Cationic Surfactants

The term “cationic surfactant” as defined by the instant disclosure is a surfactant that may be positively charged when it is contained in the hair treatment compositions according to the disclosure. The cationic surfactant may bear one or more positive permanent charges or may contain one or more functional groups that are cationizable in the composition according to the disclosure.

In a preferred embodiment, at least one of the one or more surfactants (b) is a cationic surfactant. Preferably, the hair treatment compositions includes one or more cationic surfactants and one or more surfactants chosen from nonionic surfactants, amphoteric surfactants, and a combination thereof.

Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof.

In some instances, the cationic surfactant is preferably selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof.

In some instances, the cationic surfactants are more preferably selected from cetrimonium chloride, behentrimonium chloride, behentrimonium methosulfate, stearamidopropyl dimethylamine, and a mixture thereof.

Moreover, in some cases, the cationic surfactant is most preferably cetrimonium chloride, behentrimonium chloride, or a mixture thereof.

In some embodiments, the cationic surfactant is selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, and mixtures thereof.

In some embodiments, the cationic surfactant comprises cetrimonium chloride, behentrimonium chloride, and mixtures thereof. Behentrimonium Chloride, also described by the technical names that include 1-Docosanaminium, N,N,N-Trimethyl-, Chloride, and N,N,N-Trimethyl-1-Docosanaminium Chloride, is the quaternary ammonium salt that conforms to the formula:

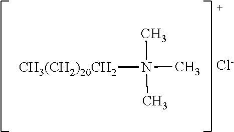

Additional nonlimiting examples of cationic surfactants include behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride(Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCI, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The cationic surfactant(s) may also be chosen from optionally polyoxyalkylenated, primary, secondary or tertiary fatty amines, or salts thereof, and quaternary ammonium salts, and mixtures thereof.

In some cases, it is useful to use salts such as chloride salts of the quaternary ammonium compounds.

The fatty amines generally comprise at least one C₈-C₃₀ hydrocarbon-based chain.

Examples of quaternary ammonium salts that may especially be mentioned include: those corresponding to the general formula below:

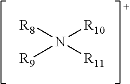

in which the groups R₈ to R₁₁, which may be identical or different, represent a linear or branched, saturated or unsaturated aliphatic group comprising from 1 to 30 carbon atoms, or an aromatic group such as aryl or alkylaryl, at least one of the groups Rs to R₁₁ denoting a group comprising from 8 to 30 carbon atoms and, in some embodiments, from 12 to 24 carbon atoms. The aliphatic groups may comprise heteroatoms especially such as oxygen, nitrogen, sulfur and halogens. The aliphatic groups are chosen, for example, from C₁-C₃₀ alkyl, C₂-C₃₀ alkenyal, C₁-C₃₀ alkoxy, polyoxy(C₂-C₆)alkylene, C₁-C₃oalkylamide, (C₁₂-C₂₂)alkylamido(C₂-C₆)alkyl, (C₁₂-C₂₂)alkyl acetate and C₁-C₃₀ hydroxyalkyl groups; Xis an anion chosen from the group of halides, phosphates, acetates, lactates, (C₁-C₄)alkyl sulfates, and (C₁-C₄)alkyl- or (C₁-C₄)alkylarylsulfonates.

Among the quaternary ammonium salts of formula (Ill), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethylammonium, cetyltrimethylammonium or benzyldimethylstearylammonium salts, or, on the other hand, oleocetyldimethylhydroxyethylammonium salts, palmitylamidopropyltrimethylammonium salts, stearamidopropyltrimethylammonium salts and stearamidopropyldimethylcetearylammonium salts.

Also useful are quaternary ammonium salts of imidazoline, such as, for example, those of formula below:

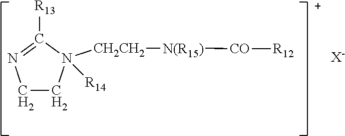

in which R₁₂ represents an alkenyl or alkyl group comprising from 8 to 30 carbon atoms, derived for example from tallow fatty acids, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, alkyl sulfates, alkyl- or alkylaryl-sulfonates in which the alkyl and aryl groups , in some embodiments, comprise, respectively, from 1 to 20 carbon atoms and from 6 to 30 carbon atoms. R₁₂ and R₁₃, in some embodiments, denote a mixture of alkenyl or alkyl groups containing from 12 to 21 carbon atoms, derived for example from tallow fatty acids, R₁₄, in some embodiments, denotes a methyl group, and R₁₅, in some embodiments, denotes a hydrogen atom. Such a product is sold, for example, under the name REWOQUAT W 75 by the company Rewo.

Useful quaternary diammonium or triammonium salts includes those of the formula:

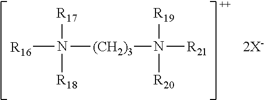

in which R₁₆denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃, R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates. Such compounds are, for example, Finquat CT-P, sold by the company Finetex (Quaternium 89), and Finquat CT, sold by the company Finetex (Quaternium 75).

Useful cationic/cationizable surfactants, including cationizable surfactants together with an acid neutralizer, include those of the general structure R₄-A-R_{5—}B wherein R₄ is a saturated or unsaturated, straight or branched alkyl chain with 8 to 24 C atoms, R₅ is a straight or branched alkyl chain with 1 to 4 C atoms, A is selected from:

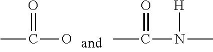

and B is selected from

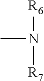

wherein R₆ and R₇ are the same or different is H or an alkyl chain with 1 to 4 C atoms, hydroxyl alkyl chain with 1 to 4 C atoms and di hydroxyl alkyl chain with 2 to 4 C atoms, and

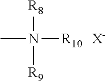

wherein R₈ and R₉ are the same or different, an alkyl chain with 1 to 4 C atoms, hydroxyl alkyl chain with 1 to 4 C atoms and di hydroxyl alkyl chain with 2 to 4 C atoms, R.sub.10 is an alkyl chain with 1 to 4 C atoms, hydroxyl alkyl chain with 1 to 4 C atoms or di hydroxyl alkyl chain with 2 to 4 C atoms.

In some instances, R₄ is saturated or unsaturated, straight or branched alkyl chain with 10 to 24C atoms, in some embodiments, 12 to 22 C atoms and R₅ is straight or branched alkyl group with 1 to 4 C atoms, and A, B, R₆ to R₁₀ are same as above.

Non-limiting suitable examples are stearyloxypropyl amine, palmityloxypropyl amine, stearyloxypropyldimethyl amine, stearyloxypropyldiethyl amine, stearyloxyethylyldimethyl amine, stearyloxyethyl amine, myristyloxypropyl amine, myristyloxypropyldimethyl amine, palmitamidopropyl amine, palmitamidopropyl methylamine, palmitamidopropyl diethylamine, palmitamidopropyl dibutylamine, palmitamidopropyl buylamine, palmitamidopropyl dipropylamine, palmitamidopropyl propylamine, palmitamidopropyl dihydroxyethylamine, palmitamidopropyl hydroxyethylamine, palmitamidopropyl dihydroxypropylamine, palmitamidopropyl hydroxypropylamine, lauramidopropyl amine, lauramidopropyl methylamine, lauramidopropyl diethylamine, lauramidopropyl dibutylamine, lauramidopropyl buylamine, lauramidopropyl dipropylamine, lauramidopropyl propylamine, lauramidopropyl dihydroxyethylamine, lauramidopropyl hydroxyethylamine, lauramidopropyl dihydroxypropylamine, lauramidopropyl hydroxypropylamine, stearamidopropyl amine, stearamidopropyl dimethylamine, steara midopropyl diethylamine, stearamidopropyldibutylamine, stearamidopropyl butylamine, stearamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, behenamidopropyl amine, behenamidopropyl methylamine, behenamidopropyl diethylamine, behenamidopropyl dibutylamine, behenamidopropyl butylamine, behenamidopropyl dipropylamine, behenamidopropyl propylamine, behenamidopropyl dihydroxyethylamine, behenamidopropyl hydroxyethylamine, behenamidopropyl dihydroxypropylamine, behenamidopropyl hydroxypropylamine, dipalmitamidopropyl methylamine, dipalmitamidopropyl ethylamine, dipalmitamidopropyl butylamine, dipalmitamidopropyl propylamine, dipalmitamidopropyl hydroxyethylamine, dipalmitamidopropyl hydroxypropylamine, dilauramidopropyl amine, dilauramidopropyl methylamine, dilauramidopropyl buylamine, dilauramidopropyl hydroxyethylamine, dilauramidopropyl hydroxypropylamine, distearamidopropyl amine, distearamidopropyl methylamine, dibehenamidopropyl propylamine, dibehenamidopropyl hydroxyethylamine, palmitoamidopropyl trimethyl ammonium chloride, stearamidopropyl trimethylammonium chloride, behenamidopropyl tri hydroxyethalmonium chloride, distearylamidopropyl dimethyl ammonium chloride, dicetylamidodihydroxyethyl ammonium chloride, palmitoylpropyl amine, palmitoylpropyl methylamine, palmitoylpropyl diethylamine, palmitoylpropyl dibutylamine, palmitoylpropyl buylamine, palmitoylpropyl dipropylamine, palmitoylpropyl propylamine, palmitoylpropyl dihydroxyethylamine, palmitoylpropyl hydroxyethylamine, palmitoylpropyl dihydroxypropylamine, palmitoylpropyl hydroxypropylamine, myristoylpropyl amine, myristoylpropyl methylamine, myristoylpropyl diethylamine, myristoylpropyl dibutylamine, myristoylpropyl buylamine, myristoylpropyl dipropylamine, myristoylpropyl propylamine, myristoylpropyl dihydroxyethylamine, myristoylpropyl hydroxyethylamine, myristoylpropyl dihydroxypropylamine, myristoylpropyl hydroxypropylamine, stearoylpropyl amine, stearoylpropyl methylamine, stearoylpropyl diethylamine, stearoylpropyl dibutylamine, stearoylpropyl butylamine, stearoylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, behenylpropyl amine, behenylpropyl methylamine, behenylpropyl diethylamine, behenylpropyl dibutylamine, behenylpropyl butylamine, behenylpropyl dipropylamine, behenylpropyl propylamine, behenylpropyl dihydroxyethylamine, behenylpropyl hydroxyethylamine, behenylpropyl dihydroxypropylamine, behenylpropyl hydroxypropylamine, dipalmitoylpropyl methylamine, dipalmitoylpropyl ethylamine, dipalmitylpropyl butylamine, dipalmitylpropyl propylamine, dipalmitylpropyl hydroxyethylamine, dipalmitylpropyl hydroxypropylamine, dilauroylpropyl amine, dilauroylpropyl methylamine, dilauroylpropyl buylamine, dilauroylpropyl hydroxyethylamine, dilauroylpropyl hydroxypropylamine, distearylpropyl amine, distearylpropyl methylamine, dibehenylpropyl propylamine, dibehenylpropyl hydroxyethylamine, palmitylpropyl trimethyl ammonium chloride, stearylpropyl trimethylammonium chloride, behenylpropyl tri hydroxyethalmonium chloride, distearylpropyl dimethyl ammonium chloride, dicetyldihydroxyethyl ammonium chloride, dioleoylethylhydroxyethylmonium methosulfate, and dicocoylethylhydroxyethylmonium methosulfate.

Cationizable surfactants may be chosen from fatty alkylamines and fatty dialkylamines. In some cases, the fatty dialkylamines may be fatty dimethylamines. Non-limiting examples include dimethyl lauramine, dimethyl behenamine, dimethyl cocamine, dimethyl myristamine, dimethyl palmitamine, dimethyl stearamine, dimethyl tallowamine, dimethyl soyamine, and mixtures thereof.

Fatty dialkylamines include fatty amidoamine compounds, their salts, and mixtures thereof. Non-limiting examples include oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, and palmitamidopropyl dimethylamine.

Non-polymeric, mono-, di-, and/or tri-carboxylic acids may be used to “neutralize” the fatty dialkylamines. In some cases, the one or more non-polymeric, mono-, di-, and/or tri-carboxylic acids include at least one dicarboxylic acid. Non-limiting examples include lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, benzoic acid, and mixtures thereof. In particular, lactic acid or tartaric acid or mixtures thereof are useful, especially in combination with fatty dimethylamines such as, for example, stearamidopropyl dimethylamine.

The total amount of the one or more cationic surfactants will vary. Nonetheless, in various embodiments, the total amount of the one or more cationic surfactants is from about 1 to about 10 wt. %, based on the total weight of the hair treatment composition.

In further embodiments, the total amount of the one or more cationic surfactants is from about 1 to about 8 wt. %, about 1 to about 6 wt. %, about 2 to about 10 wt. %, about 2 to about 8 wt. %, about 2 to about 6 wt %, about 3 to about 10 wt. %, about 3 to about 8 wt. %, or about 3 to about 6 wt. %, about 3.5 to about 10 wt. %, about 3.5 to about 8 wt. %, about 3.5 to about 6 wt. %, about 3.8, 4, 4.1, 4.2, 4.5, 4.6, 4.8, 5, 5.2, including any combination, sub-combination, range, or sub-range thereof by weight, based on the total weight of the hair treatment composition.

(ii) Nonionic Surfactants,

In various embodiments, the hair treatment compositions include one or more nonionic surfactants. Nonionic surfactants can be useful for enhancing emulsification.

Therefore, nonionic surfactants may also be referred to as nonionic emulsifiers. For example, the nonionic surfactants include surfactants/emulsifier that are useful for forming an emulsion, for example, oil-in-water emulsions and vice versa. Nonlimiting examples of nonionic surfactants include alkyl polyglucosides; alcohols, alpha-diols, alkylphenols and esters of fatty acids, being ethoxylated, propoxylated or glycerolated (polyglyceryl-2 isostearate); ethoxylated fatty esters; glyceryl esters of fatty acids; fatty alcohol ethoxylates; alkyl phenol ethoxylates; fatty acid alkoxylates; and mixtures thereof.

Nonlimiting examples of polyglycerolated fatty acid esters include polyglyceryl-2 laurate, polyglyceryl-3 laurate, polyglyceryl-4 laurate, polyglyceryl-5 laurate, polyglyceryl-6 laurate, polyglyceryl-10 laurate; polyglyceryl-2 myristate, polyglyceryl-3 myristate, polyglyceryl-4 myristate, polyglyceryl-5 myristate, polyglyceryl-6 myristate, polyglyceryl-10 myristate; polyglyceryl-2 palmitate, polyglyceryl-3 palmitate, polyglyceryl-6 palmitate, polyglyceryl-10 palmitate, polyglyceryl-2 isostearate, polyglyceryl-3 isostearate, polyglyceryl-4 isostearate, polyglyceryl-5 isostearate, polyglyceryl-6 isostearate, polyglyceryl-10 isostearate; polyglyceryl-2 stearate, polyglyceryl-3 stearate, polyglyceryl-4 stearate, polyglyceryl-5 stearate, polyglyceryl-6 stearate, polyglyceryl-8 stearate, polyglyceryl-10 stearate, and mixtures thereof. In some instances, polyglyceryl-2 isostearate is particularly useful.

The nonionic surfactants/emulsifiers may be chosen from alcohols and alpha-diols, these compounds being polyethoxylated and/or polypropoxylated and/or polyglycerolated, the number of ethylene oxide and/or propylene oxide groups possibly ranging from 2 to 100, and the number of glycerol groups possibly ranging from 2 to 30; these compounds comprising at least one fatty chain comprising from 8 to 30 carbon atoms and especially from 16 to 30 carbon atoms.

Mention is also be made of polyethoxylated fatty amides preferably having from 2 to 30 ethylene oxide units, polyglycerolated fatty amides including on average from 1 to 5, and in particular from 1.5 to 4, glycerol groups; polyoxyethylenated fatty acid esters of sorbitan having preferably from 2 to 40 units of ethylene oxide, fatty acid esters of sucrose, polyoxyalkylenated and preferably polyoxyethylenated fatty acid esters containing from 2 to 150 mol of ethylene oxide, such as oxyethylenated plant oils.

Useful nonionic surfactants include those of the alkyl(poly)glycoside type, represented especially by the following general formula: R₁O—(R₂O)ₜ-(G)ᵥ in which: R₁ represents a linear or branched alkyl or alkenyl substituent comprising 6 to 24 carbon atoms and especially 8 to 18 carbon atoms, or an alkylphenyl substituent whose linear or branched alkyl substituent comprises 6 to 24 carbon atoms and especially 8 to 18 carbon atoms; R₂ represents an alkylene substituent comprising 2 to 4 carbon atoms; G represents a sugar unit comprising 5 to 6 carbon atoms; t denotes a value ranging from 0 to 10 and preferably 0 to 4; and v denotes a value ranging from 1 to 15 and preferably 1 to 4. Preferably, the alkyl(poly)glycoside surfactants are compounds of the formula described above in which: R₁ denotes a linear or branched, saturated or unsaturated alkyl substituent comprising from 8 to 18 carbon atoms; R₂ represents an alkylene substituent comprising 2 to 4 carbon atoms; t denotes a value ranging from 0 to 3 and preferably equal to 0; and G denotes glucose, fructose or galactose, preferably glucose; the degree of polymerization, i.e. the value of v, possibly ranging from 1 to 15 and preferably from 1 to 4; the mean degree of polymerization more particularly being between 1 and 2. The glucoside bonds between the sugar units are generally of 1-6 or 1-4 type and preferably of 1-4 type. In particular, the alkyl(poly)glycoside surfactant may be an alkyl(poly)glucoside surfactant C₈/C₁₆ alkyl(poly)glucosides 1,4, and in particular decyl glucosides and caprylyl/capryl glucosides.

Useful nonionic surfactants may be chosen from polyoxyethylenated C8-C30 fatty acid esters (preferably C12-C18) of sorbitan, polyethoxylated C8-C30 (preferably C12-18) fatty alcohols, polyglycerolated C8-C30 (preferably C12-C18) fatty acid esters, polyoxyethylenated compounds having preferably from 2 to 30 moles of ethylene oxide, polyglycerolated compounds having preferably from 2 to 16 moles of glycerol; and mixtures thereof.

The polyoxyethylenated C8-C30 fatty alcohols may be chosen from C12-C18 fatty alcohols, in particular polyoxyethylenated lauryl alcohol, cetyl alcohol, myristyl alcohol, and stearyl alcohol having from 2 to 30 mol of ethylene oxide, such as: cetyl alcohol polyoxyethylenated with 2 EO (Ceteth-2) (HLB 5.3) cetyl alcohol polyoxyethylenated with 6 EO (Ceteth-6) (HLB 11.1) cetyl alcohol polyoxyethylenated with 10 EO (Ceteth-10) (HLB 12.9) cetyl alcohol polyoxyethylenated with 20 EO (Ceteth-20) (HLB 15.7) cetyl alcohol polyoxyethylenated with 24 EO (Ceteth-24) (HLB 16.3) lauryl alcohol polyoxyethylenated with 2 EO (Laureth-2) (HLB 6.1) lauryl alcohol polyoxyethylenated with 3 EO (Laureth-3) (HLB 8) lauryl alcohol polyoxyethylenated with 4 EO (Laureth-4) (HLB 9.4) lauryl alcohol polyoxyethylenated with 7 EO (Laureth-7) (HLB 12.3) lauryl alcohol polyoxyethylenated with 9 EO (Laureth-9) (HLB 13.6) lauryl alcohol polyoxyethylenated with 10 EO (Laureth-10) (HLB 13.9) lauryl alcohol polyoxyethylenated with 12 EO (Laureth-12) (HLB 14.6) lauryl alcohol polyoxyethylenated with 21 EO (Laureth-21) (HLB 15.5) lauryl alcohol polyoxyethylenated with 23 EO (Laureth-23) (HLB 16.3) stearyl alcohol polyoxyethylenated with 2 EO (Steareth-2) (HLB 4.9) stearyl alcohol polyoxyethylenated with 10 EO (Steareth-10) (HLB 12.4) stearyl alcohol polyoxyethylenated with 20 EO (Steareth-20) (HLB 15.2) stearyl alcohol polyoxyethylenated with 21 EO (Steareth-21) (HLB 15.5)

The polyoxyethylenated C8-C30 fatty acid esters (preferably C12-C18) of sorbitan may be chosen from polyoxyethylenated esters of C12-C18 fatty acids, in particular lauric, myristic, cetylic or stearic acids, of sorbitan especially containing from 2 to 30 mol of ethylene oxide, such as: polyoxyethylenated sorbitan monolaurate (4 EO) (Polysorbate-21) (HLB 13.3) polyoxyethylenated sorbitan monolaurate (20 EO) (Polysorbate-20) (HLB 16.7) polyoxyethylenated sorbitan monopalmitate (20 EO)

(Polysorbate-40) (HLB 15.6) polyoxyethylenated sorbitan monostearate (20 EO) (Polysorbate-60) (HLB 14.9) polyoxyethylenated sorbitan monostearate (4 EO) (Polysorbate-61) (HLB 9.6) polyoxyethylenated sorbitan monooleate (20 EO) (Polysorbate-80) (HLB 15). In a preferred embodiment, the hair treatment compositions include one or more nonionic surfactants chosen from polyoxyethylenated C8-C30 fatty acid esters (preferably C12-C18) of sorbitan, preferably polyoxyethylenated esters of C12-C18 fatty acids.

The polyglycerolated C8-C30 fatty acid esters, which are particularly preferred, may be chosen from polyglycerolated esters of C12-C18 fatty acids, in particular lauric, myristic, palmitic, stearic or isostearic acid, having from 2 to 16 mol of glycerol, such as:

polyglyceryl-2 laurate, polyglyceryl-3 laurate, polyglyceryl-4 laurate, polyglyceryl-5 laurate, polyglyceryl-6 laurate, polyglyceryl-10 laurate; polyglyceryl-2 myristate, polyglyceryl-3 myristate, polyglyceryl-4 myristate, polyglyceryl-5 myristate, polyglyceryl-6 myristate, polyglyceryl-10 myristate; polyglyceryl-2 palmitate, polyglyceryl-3 palmitate, polyglyceryl-6 palmitate, polyglyceryl-10 palmitate; polyglyceryl-2 isostearate, polyglyceryl-3 isostearate, polyglyceryl-4 isostearate, polyglyceryl-5 isostearate, polyglyceryl-6 isostearate, polyglyceryl-10 isostearate; polyglyceryl-2 stearate, polyglyceryl-3 stearate, polyglyceryl-4 stearate, polyglyceryl-5 stearate, polyglyceryl-6 stearate, polyglyceryl-8 stearate, polyglyceryl-10 stearate, and mixtures thereof.

In some cases, the nonionic surfactant may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

As glyceryl esters of fatty acids mention is made of glyceryl stearate (glyceryl mono-, di- and/or tristearate) (CTFA name: glyceryl stearate) or glyceryl ricinoleate and mixtures thereof.

As glyceryl esters of C₈⁻C₂₄ alkoxylated fatty acids, polyethoxylated glyceryl stearate (glyceryl mono-, di- and/or tristearate) such as PEG-20 glyceryl stearate can for example be cited.

Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEG1N by Goldschmidt (CTFA name: glyceryl stearate SE), can also be used.

Alkyl polyglucosides are a class of useful nonionic surfactants. Non-limiting examples of alkyl polyglucosides include alkyl polyglucosides having the following formula:

R¹—O—(R²O)ₙ—Z(x)

wherein R¹ is an alkyl group having 8-18 carbon atoms;

R² is an ethylene or propylene group;

Z is a saccharide group with 5 to 6 carbon atoms;

n is an integer from 0 to 10; and

x is an integer from 1 to 5.

Useful alkyl poly glucosides include lauryl glucoside, octyl glucoside, decyl glucoside, coca glucoside, sucrose laurate, caprylyl/capryl glucoside, and sodium lauryl glucose carboxylate, and mixtures thereof. Typically, the at least one alkyl poly glucoside compound is selected from the group consisting of lauryl glucoside, decyl glucoside and coca glucoside, and more typically lauryl glucoside.

The total amount of nonionic, if present, will vary. Nonetheless, when present, the total amount of nonionic surfactants is from about 0.01 to about 10 wt. %, based on the total weight of the hair treatment composition. In certain embodiments, the total amount of the one or more nonionic surfactants is from about 0.01 to about 8 wt. %, about 0.01 to about 5 wt. %, about 0.01 to about 3 wt. %, about 0.01 to about 2 wt. %, about 0.05 to about 10 wt. %, about 0.05 to about 8 wt. %, about 0.05 to about 5 wt. %, about 0.05 to about 3 wt. %, about 0.05 to about 2 wt. %, about 0.1 to about 10 wt. %, about 0.1 to about 8 wt. %, about 0.1 to about 5 wt. %, about 0.1 to about 3 wt. %, about 0.1 to about 2 wt. %, about 0.1 to about 1 wt. %, including any combination, sub-combination, range, or sub-range thereof by weight, based on the total weight of the hair treatment composition.

(iii) Amphoteric Surfactants

Nonlimiting examples of useful amphoteric surfactants include betaines, alkyl sultaines, alkyl amphoacetates, alkyl amphoproprionates, and mixtures thereof, as provided below. In certain embodiments, betaines are particularly useful.

(iii-a) Betaines

Useful betaines include those of the following formulae (XIIIa-XIIId):

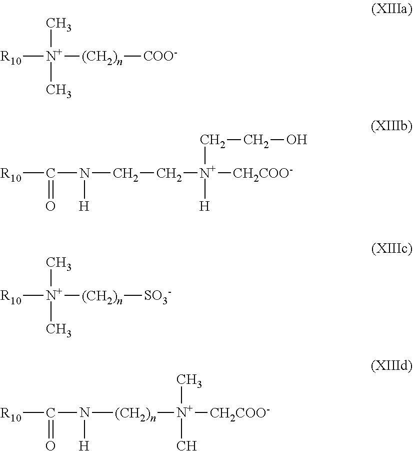

wherein R₁₀ is an alkyl group having 8-18 carbon atoms; and n is an integer from 1 to 3.

Particularly useful betaines include, for example, coca betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, and mixtures thereof. Typically, at least one betaine compound is selected from coco betaine, behenyl betaine, capryl/capramidopropyl betaine, and lauryl betaine, and mixtures thereof. Particularly preferred betaines include coco betaine and cocamidopropyl betaine.

(iii-b) Alkyl Sultaines

Non-limiting examples of alkyl sultaines include hydroxyl sultaines of formula (XIV)

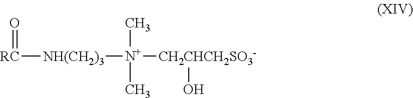

wherein R is an alkyl group having 8-18 carbon atoms. More specific examples include, but are not limited to cocamidopropyl hydroxysultaine, lauryl hydroxysultaine, and a mixture thereof.

(iii-c) Alkyl Amphoacetates and Alkyl Amphodiacetates

Nonlimiting examples of alkyl amphoacetates and alkyl amphodiacetates include those of Formula (XV) and (XVI):

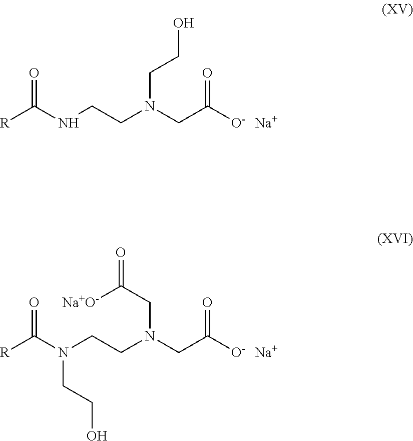

wherein R is an alkyl group having 8-18 carbon atoms. Sodium is shown as the cation in the above formulae but the cation may be an alkali metal ion such as sodium or potassium, ammonium ions, or alkanolammonium ions such as monoethanolammonium or triethanolammonium ions. A more specific, but non-limiting example, is sodium lauroamphoacetate.

(iii-d) Alkyl Amphopropionates

Non-limiting examples of amphopropionates include cocoamphopropionate, caprylamphopropionate, cornamphopropionate,caproamphopropionate, oleoamphopropionate, isostearoamphopropionate, stearoamphopropionate, lauroamphopropionate, salts thereof, and a mixture thereof.

The total amount of amphoteric surfactants in the hair treatment compositions, if present, will vary. Nonetheless, in various embodiments, the total amount of the one or more amphoteric surfactants in the hair treatment compositions is from about 0.01 to about 10 wt. %, based on the total weight of the hair treatment composition. In certain embodiments, the total amount of the one or more amphoteric surfactants is from about 0.01 to about 8 wt. %, about 0.01 to about 5 wt. %, about 0.01 to about 3 wt. %, about 0.01 to about 2 wt. %, about 0.01 to about 1 wt. %, about 0.05 to about 10 wt. %, about 0.05 to about 8 wt. %, about 0.05 to about 5 wt. %, about 0.05 to about 3 wt. %, about 0.05 to about 2 wt. %, about 0.05 to about 1 wt. %, about 0.1 to about 10 wt. %, about 0.1 to about 8 wt. %, about 0.1 to about 5 wt. %, about 0.1 to about 3 wt. %, about 0.1 to about 2 wt. %, about 0.1 to about 1 wt. %, including any combination, sub-combination, range, or sub-range thereof by weight, based on the total weight of the hair treatment composition.

(iv) Anionic Surfactants

In various embodiments, the hair treatment compositions include one or more anionic surfactants. Nonetheless, in other embodiments, the hair treatment compositions are free or essentially free from anionic surfactants, for example, in certain embodiments, the hair treatment compositions comprise less than 5 wt. %, less than 4 wt. %, less than 3 wt. %, less than 2 wt. %, less than 1 wt. %, less than 0.5 wt. %, less than 0.1 wt. % of one or more anionic surfactants.

Anionic surfactants can be sulfate based or non-sulfate based. Nonlimiting examples of sulfate based anionic surfactants include alkyl sulfates and alkyl ether sulfates. Nonlimiting examples of non-sulfate based anionic surfactants include alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof.

Useful alkyl sulfates include C₈₋₁₈ alky sulfates, more preferably C₁₂₋₁₈ alkyl sulfates, preferably in the form of a salt with a solubilizing cation such as sodium, potassium, ammonium or substituted ammonium. Examples are sodium lauryl sulfate (SLS) or sodium dodecyl sulfate (SDS). Useful alkyl ether sulfates include those having the formula: RO(CH₂CH₂O)ₙSO₃M; wherein R is an alkyl or alkenyl having from 8 to 18 (preferably 12 to 18) carbon atoms; n is a number having an average value of greater than at least 0.5, preferably between 1 and 3, more preferably between 2 and 3; and M is a solubilizing cation such as sodium, potassium, ammonium or substituted ammonium. An example is sodium lauryl ether sulfate (SLES).

In some instances, useful alkyl sulfate salts and alkyl ether sulfate salts include those having the formulas (I and II):

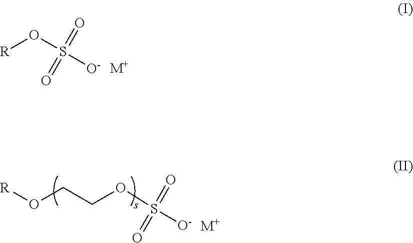

wherein, R is alkyl chain having 6 to 24 carbon atoms, preferably 8 to 18 carbon atoms, and more preferably 12 to 18 carbon atoms;

M is a solubilizing cation such as alkali metal ions such as sodium or potassium, ammonium ions, or alkanolammonium ions such as monoethanolammonium or triethanolammonium ions; and

n is an integer from 0 to 3.

As already noted, nonlimiting examples of non-sulfate based anionic surfactants include alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof. Nonlimiting examples of various types of non-sulfate based anionic surfactants are provided below.

Alkyl Sulfonates

Useful alkyl sulfonates include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (III) is particularly useful.

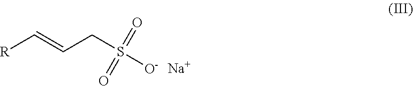

R is selected from H or alkyl chain that has 1-24 carbon atoms, preferably 6-24 carbon atoms, more preferably, 8 to 20 carbon atoms, said chain being saturated or unsaturated, linear or branched. Sodium is shown as the cation in the above formula (III) but the cation may be an alkali metal ion such as sodium or potassium, ammonium ions, or alkanolammonium ions such as monoethanolammonium or triethanolammonium ions. In some instances, the alkyl sulfonate(s) are selected from C₈-C₁₆ alkyl benzene sulfonates, C₁₀-C₂₀ paraffin sulfonates, C₁₀-C₂₄ olefin sulfonates, salts thereof, and mixtures thereof. C₁₀-C₂₄ olefin sulfonates are particularly preferred. A non-limiting but particularly useful example of a C₁₀-C₂₄ olefin sulfonate that can be used in the instant compositions is sodium C₁₄₋₁₆ olefin sulfonate.

Alkyl Sulfosuccinates

Non-limiting examples of useful sulfosuccinates include those of formula (IV):

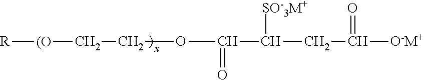

wherein R is a straight or branched chain alkyl or alkenyl group having 10 to 22 carbon atoms, preferably 10 to 20 carbon atoms, X is a number that represents the average degree of ethoxylation and can range from 0 to about 5, preferably from 0 to about 4, and most preferably from about 2 to about 3.5, and M and M′ are monovalent cations which can be the same or different from each other. Preferred cations are alkali metal ions such as sodium or potassium, ammonium ions, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, and a mixture thereof. In some instances, disodium laureth sulfosuccinate is particularly preferred.

Alkyl Sulfoacetates

Non-limiting examples of alkyl sulfacetates includes, for example, alkyl sulfoacetates such as C₄-C₁₈ fatty alcohol sulfoacetates and/or salts thereof. A particularly preferred sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

Acyl Isethionates

Non-limiting examples of useful acyl isethionates include those of formula (V) and (VI):

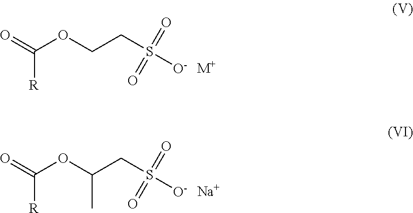

wherein R, R¹, R² and R³ are each independently selected from H or an alkyl chain having 1-24 carbon atoms, said chain being saturated or unsaturated, linear or branched, and X is COO⁻ or SO₃⁻. Sodium is shown as the cation in formula (VI) but the cation for both formula (V) and formula (VI) may be an alkali metal ion such as sodium or potassium, ammonium ions, or alkanolammonium ions such as monoethanolammonium or triethanolammonium ions. Non-limiting examples of acyl isethionates include sodium isethionate, sodium cocoyl isethionate, sodium lauroyl methyl isethionate, and sodium cocoyl methyl isethionate.

Alkoxylated Monoacids

Non-limiting examples of alkoxylated monoacids include compounds corresponding to formula (VII):

RO[CH2O]u[(CH2)xCH(R′)(CH2)y(CH2)zO]v[CH2CH2O]wCH2COOH

wherein:

R is a hydrocarbon radical containing from about 6 to about 40 carbon atoms;

u, v and w, independently of one another, represent numbers of from 0 to 60;

x, y and z, independently of one another, represent numbers of from 0 to 13;

R′ represents hydrogen, alkyl, and

the sum of x+y+z>0;

Compounds corresponding to formula (VII) can be obtained by alkoxylation of alcohols ROH with ethylene oxide as the sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (VII), R is linear or branched, acyclic or cyclic, saturated or unsaturated, aliphatic or aromatic, substituted or unsubstituted. Typically, R is a linear or branched, acyclic C6-40 alkyl or alkenyl group or a C1-40 alkyl phenyl group, more typically a C8-22 alkyl or alkenyl group or a C4-18 alkyl phenyl group, and even more typically a C12-18 alkyl group or alkenyl group or a C6-16 alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C9-11 Pareth-6 Carboxylic Acid, C11-15 Pareth-7 Carboxylic Acid,

C12-13 Pareth-5 Carboxylic Acid, C12-13 Pareth-8 Carboxylic Acid, C12-13 Pareth-12 Carboxylic Acid, C12-15 Pareth-7 Carboxylic Acid, C12-15 Pareth-8 Carboxylic Acid, C14-15 Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof. In some cases, preferred ethoxylated acids include Oleth-10 Carboxylic Acid, Laureth-5 Carboxylic Acid, Laureth-11 Carboxylic Acid, and a mixture thereof.

Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt. Non-limiting examples of useful acyl amino acids include those of formula (VIII):

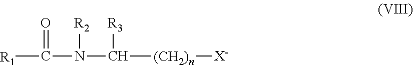

wherein R, R¹, R² and R³ are each independently selected from H or an alkyl chain having 1-24 carbon atoms, said chain being saturated or unsaturated, linear or branched, and X is COO⁻ or SO₃⁻.

Acyl Taurates: Non-limiting examples of acyl taurates include those of formula (IX):

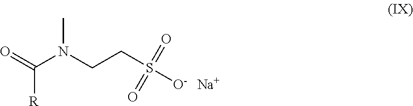

wherein R, R¹, R² and R³ are each independently selected from H or an alkyl chain having 1-24 carbon atoms, or from 6-20 carbon atoms, or from 8 to 16 carbon atoms, said chain being saturated or unsaturated, linear or branched, and X is COO⁻ or SO₃⁻. Non-limiting examples of acyl taurate salts include sodium cocoyl taurate and sodium methyl cocoyl taurate.

Acyl Glycinates: Non-limiting examples of useful acyl glycinates include those of formula (X):

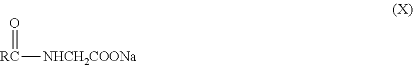

wherein R is an alkyl chain of 8 to 16 carbon atoms. Sodium is shown as the cation in the above formula (X) but the cation may be an alkali metal ion such as sodium or potassium, ammonium ions, or alkanolammonium ions such as monoethanolammonium or triethanolammonium ions. Non-limiting examples of acyl glycinates include sodium cocoyl glycinate, sodium lauroyl glycinate, sodium myristoyl glycinate, potassium lauroyl glycinate, and potassium cocoyl glycinate, and in particular sodium cocoyl glycinate.

Acyl Glutamates: Non-limiting examples of useful acyl glutamates include those of formula (XI):

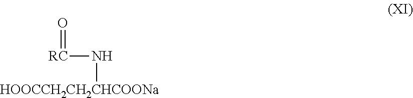

wherein R is an alkyl chain of 8 to 16 carbon atoms. Sodium is shown as the cation in the above formula (XI) but the cation may be an alkali metal ion such as sodium or potassium, ammonium ions, or alkanolammonium ions such as monoethanolammonium or triethanolammonium ions. Non-limiting examples of acyl gluatamtes include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate. In some cases, sodium stearoyl glutamate is particularly preferred.

Acyl Sarcosinates: Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

The total amount of the one or more anionic surfactants, if present, will vary. Nonetheless, in various embodiments, the total amount of the one or more anionic surfactants is from about 0.01 to about 15 wt. %, based on the total weight of the hair treatment composition. In certain embodiments, the total amount of the one or more anionic surfactants is from about 0.01 to about 10 wt. %, about 0.01 to about 8 wt. %, about 0.01 to about 5 wt. %, about 0.01 to about 3 wt. %, about 0.01 to about 1 wt. %, about 0.01 to about 0.5 wt. %, about 0.05 to about 15 wt. %, about 0.05 to about 10 wt. %, about 0.05 to about 8 wt. %, about 0.05 to about 5 wt. %, about 0.05 to about 3 wt. %, about 0.05 to about 1 wt. %, about 0.05 to about 0.5 wt. %, about 0.1 to about 15 wt. %, about 0.1 to about 10 wt. %, about 0.1 to about 8 wt. %, about 0.1 to about 5 wt. %, about 0.1 to about 3 wt. %, about 0.1 to about 1 wt. %, including any combination, sub-combination, range, or sub-range thereof by weight, based on the total weight of the hair treatment composition.

(c) Water

The total amount of water in the hair treatment compositions can and will vary depending on the amount of the other components in the hair treatment compositions. Nonetheless, in various embodiments, the total amount of water in the hair treatment composition is from about 50 to about 90 wt. %, based on the total weight of the hair treatment composition. In further embodiments, the total amount of water is from about 50 to about 85 wt. %, about 50 to about 80 wt. %, about 55 to about 90 wt. %, about 55 to 85 wt. %, about 55 to about 80 wt. %, about 60 to about 90 wt. %, about 60 to 85 wt. %, about 60 to about 80 wt. %, about 65 to about 90 wt. %, about 65 to about 85 wt. %, about 65 to about 80 wt. %, including any combination, sub-combination, range, or sub-range thereof by weight, based on the total weight of the hair treatment composition.

(d) Fatty Alcohols

The term “fatty alcohol” means an alcohol comprising at least one hydroxyl group (OH), and comprising at least 8 carbon atoms, and which is neither oxyalkylenated (in particular neither oxyethylenated nor oxypropylenated) nor glycerolated. The fatty alcohols can be represented by: R-OH, wherein R denotes a saturated (alkyl) or unsaturated (alkenyl) group, linear or branched, comprising from 8 to 40 carbon atoms, preferably 10 to 30 carbon atoms, more preferably 12 to 24 carbon atoms, and even more preferably 14 to 22 carbon atoms.

The fatty alcohol(s) may be liquid or solid. In some instances, it is preferable that the cosmetic compositions include at least one solid fatty alcohol. The solid fatty alcohols that can be used include those that are solid at ambient temperature and at atmospheric pressure (25° C., 780 mmHg), and are insoluble in water, that is to say they have a water solubility of less than 1% by weight, preferably less than 0.5% by weight, at 25° C., 1 atm.

The solid fatty alcohols may be represented by: R—OH, wherein R denotes a linear alkyl group, optionally substituted with one or more hydroxyl groups, comprising from 8 to 40 carbon atoms, preferably 10 to 30 carbon atoms, more preferably 12 to 24 carbon atoms, and even more preferably 14 to 22 carbon atoms.

Non-limiting examples of useful fatty alcohols include lauryl alcohol or lauryl alcohol (1-dodecanol); myristic or myristyl alcohol (1-tetradecanol); cetyl alcohol (1-hexadecanol); stearyl alcohol (1-octadecanol); arachidyl alcohol (1-eicosanol); behenyl alcohol (1-docosanol); lignoceryl alcohol (1-tetracosanol); ceryl alcohol (1-hexacosanol); montanyl alcohol (1-octacosanol); myricylic alcohol (1-triacontanol), and mixtures thereof.

In certain embodiments, the one or more fatty alcohols have from 12 to 24 carbon atoms. Specific nonlimiting examples include cetyl alcohol, stearyl alcohol, cetearyl alcohol, behenyl alcohol, lauryl alcohol, myristic or myristyl alcohol, arachidyl alcohol, lignoceryl alcohol, or mixtures thereof.

Preferably, the cosmetic composition includes one or more solid fatty alcohols, for example, chosen from cetyl alcohol, stearyl alcohol, behenyl alcohol and mixtures thereof, preferably cetyl alcohol, behenyl alcohol, cetearyl alcohol, and mixtures thereof.

The liquid fatty alcohols, in particular those containing C10-C34, preferably have branched carbon chains and/or have one or more, preferably 1 to 3 double bonds. They are preferably branched and/or unsaturated (C═C double bond) and contain from 12 to 40 carbon atoms.

The liquid fatty alcohols may be represented by: R—OH, wherein R denotes a C12-C24 branched alkyl group or an alkenyl group (comprising at least one C12-C24 double bond C═C), R being optionally substituted by a or more hydroxy groups. Preferably, the liquid fatty alcohol is a branched saturated alcohol. Preferably, R does not contain a hydroxyl group. These include oleic alcohol, linoleic alcohol, linolenic alcohol, isocetyl alcohol, isostearyl alcohol, 2-octyl-1-dodecanol, 2-butyloctanol, 2-hexyl-1-decanol, 2-decyl-1-tetradecanol, 2-tetradecyl-1-cetanol and mixtures thereof. Preferably, the liquid fatty alcohol is 2-octyl-1-dodecanol.

In some instances, the cosmetic compositions include one or more fatty alcohols selected from decyl alcohol, undecyl alcohol, dodecyl, myristyl, cetyl alcohol, stearyl alcohol, cetearyl alcohol, isostearyl alcohol, isocetyl alcohol, behenyl alcohol, linalool, oleyl alcohol, myricyl alcohol and a mixture thereof. In some instances, the cosmetic compositions preferably include cetyl alcohol, behenyl alcohol, and cetearyl alcohol.

The total amount of the one or more fatty alcohols will vary. Nonetheless, in various embodiments, the total amount of the one or more fatty alcohols in the hair treatment compositions is from 1 to about 20 wt. %, based on the total weight of the hair treatment composition. In further embodiments, the total amount of one or more fatty alcohols in the hair treatment compositions is from about 1 to about 18 wt. %, about 1 to about 15 wt. %, about 2 to about 20 wt. %, about 2 to about 18 wt. %, about 2 to about 15 wt. %, about 3 to about 20 wt. %, about 3 to about 18 wt. %, about 3 to about 15 wt. %, about 5 to about 20 wt. %, about 5 to about 18 wt. %, about 5 to about 15 wt. %, about 7 to about 20 wt. %, about 7 to about 18 wt. %, about 7 to about 15 wt. %, about 8, 9, 10, 11, 12, 13, or 14 wt. %, including any combination, sub-combination, range, or sub-range thereof by weight, based on the total weight of the hair treatment composition.

(e) Fatty Compounds Other than Fatty Alcohols

The term “fatty compounds” is interchangeable with the “fatty materials.” Fatty compounds are known as compounds that are not soluble (or only sparingly soluble) in water; they are hydrophilic and are often solubilized in organic solvents. They include materials such as oils, fats, waxes, hydrocarbons, fatty alcohols, fatty acids, fatty esters, etc. However, for purposes of the instant disclosure fatty alcohols are not included as a fatty compound. Fatty alcohols are separately identified. Furthermore, for purposes of the instant disclosure, silicones are not considered fatty compounds. Non-limiting examples of useful fatty compounds (other than fatty alcohols) include oils, waxes, alkanes (paraffins), fatty alcohols, fatty acids, fatty esters, triglyceride compounds, lanolin, hydrocarbons, derivatives thereof, and mixtures thereof. Fatty compounds are described by the International Federation Societies of Cosmetic Chemists, for example, in Cosmetic Raw Material Analysis and Quality, Volume I: Hydrocarbons, Glycerides, Waxes and Other Esters (Redwood Books, 1994), which is incorporated herein by reference in its entirety.

Non-limiting examples of fatty compounds other than fatty alcohols include oils, mineral oil, alkanes (paraffins), fatty acids, fatty alcohol derivatives, fatty acid derivatives, esters of fatty alcohols, hydroxy-substituted fatty acids, waxes, triglyceride compounds, lanolin, and a mixture thereof.

Fatty Alcohol Derivatives

Fatty alcohol derivatives include fatty esters derived from one or more fatty alcohols. Fatty alcohol derivatives also include alkoxylated fatty alcohols, e.g., having about 1 to about 100 moles of an alkylene oxide per mole of alkoxylated fatty alcohol. For example, the alkoxylated fatty alcohols may be alkoxylated with about 1 to about 80 moles, about 2 to about 50, about 5 to about 45 moles, about 10 to about 40 moles, or 15 to about 35 mores, including all ranges and subranges therebetween, of an alkylene oxide per mole of alkoxylated fatty alcohol.

As examples of alkoxylated fatty alcohols, steareth (for example, steareth-2, steareth-20, and steareth-21), laureth (for example, laureth-4, and laureth-12), ceteth (for example, ceteth-10 and ceteth-20) and ceteareth (for example, ceteareth-2, ceteareth-10, and ceteareth-20) are mentioned. In at least one instance, the one or more alkoxylated fatty alcohols include steareth-20. In some instances, the one or more alkoxylated fatty alcohols may be exclusively steareth-20.

Additional fatty alcohol derivatives that may, optionally be suitable include methyl stearyl ether; 2-ethylhexyl dodecyl ether; stearyl acetate; cetyl propionate; the ceteth series of compounds, such as ceteth-1 through ceteth-45, which are ethylene glycol ethers of cetyl alcochol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof.

Fatty Acids

In some instances, the fatty compounds may be chosen from fatty acids, fatty acid derivatives, esters of fatty acids, hydroxyl-substituted fatty acids, and alkoxylated fatty acids. The fatty acids may be straight or branched chain acids and/or may be saturated or unsaturated. Non-limiting examples of fatty acids include diacids, triacids, and other multiple acids as well as salts of these fatty acids. For example, the fatty acid may optionally include or be chosen from lauric acid, palmitic acid, stearic acid, behenic acid, arichidonic acid, oleic acid, isostearic acid, sebacic acid, and a mixture thereof. In some cases, the fatty acids are selected from the group consisting of palmitic acid, stearic acid, and a mixture thereof.

Non-limiting examples of polyglycerol esters of fatty acids include those of the following formula:

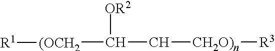

wherein the average value of n is about 3 and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀.

The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substituted fatty acids, and a mixture thereof. Non-limiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof. Preferred for use herein are glycerol monostearate, 12-hydroxy stearic acid, and a mixture thereof.

Waxes

The fatty compounds may, in some instances, include or be chosen from one or more waxes. Non-limiting examples of waxes in this category include for example, synthetic wax, ceresin, paraffin, ozokerite, polyethylene waxes, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, acacia decurrents flower wax, vegetable waxes (such as sunflower seed (helianthus annuus), carnauba, candelilla, ouricury or japan wax or cork fibre or sugarcane waxes), or a mixture thereof.

Oils

In some instances, the fatty compounds may include or be chosen from one or more oil(s). Suitable oils include, but are not limited to, natural oils, such as coconut oil;

hydrocarbons, such as mineral oil and hydrogenated polyisobutene; fatty alcohols, such as octyldodecanol; esters, such as C₁₂-C₁₅ alkyl benzoate; diesters, such as propylene dipelarganate; and triesters, such as glyceryl trioctanoate. Non-limiting examples of oils that may, optionally, be included in the cosmetic compositions include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, castor oil, lanolin and lanolin derivatives, triisocetyl citrate, sorbitan sesquioleate, C₁₀-C₁₈ triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof.

In some embodiments, the cosmetic composition may include one or more fatty compounds chosen from fatty esters (such as isopropyl myristate, cetyl esters, isononyl isononanoate), polyolefins (such as petrolatum), waxes (for example, candelilla wax and the like), squalene, squalane, hydrogenated polyisobutene, hydrogenated polydecene, polybutene, mineral oil, pentahydrosqualene, plant and/or vegetable oil, hydrocarbon-based oils (such as isohexadecane), or a mixture thereof.

The total amount of the one or more fatty compounds will vary. Nonetheless, in various embodiments, the total amount of the one or more fatty compounds is from about 1 to about 25 wt. %, based on the total weight of the hair treatment compositions.

In certain embodiments, the total amount of the one or more fatty compounds is from about 0.5 to about 20 wt. %, about 0.5 to about 15 wt. %, about 0.5 to about 10 wt. %, about 0.5 to about 8 wt. %, about 0.5 to about 5 wt. %, about 1 to about 20 wt. %, about 1 to about 15 wt. %, about 1 to about 10 wt. %, about 1 to about 8 wt. %, about 1 to about 5 wt. %, about 1 to about 4 wt. %, about 1 to about 3 wt. %, including any combination, sub-combination, range, or sub-range thereof by weight, based on the total weight of the hair treatment composition.

Ratio of Fatty Alcohol to Fatty Compounds

The weight ratio of the total amount of the one or more fatty alcohols to the total amount of the one or more fatty compounds (other than the one or more fatty alcohols) will vary. Nonetheless, in various embodiments, the weight ratio is from about 2:1 to about 15:1 ((fatty alcohols(s):(fatty compounds)). In certain embodiments, the weight ratio is from about 2:1 to about 12:1, about 2:1 to about 10:1, about 2:1 to about 8:1, about 2:1 to about 7:1, about 3:1 to about 15:1, about 3:1 to about 12:1, about 3:1 to about 10:1, about 3:1 to about 8:1, about 3:1 to about 7:1, about 4:1 to about 15:1, about 4:1 to about 12:1, about 4:1 to about 10:1, about 4:1 to about 8:1, about 4:1 to about 7:1, about 5:1 to about 15:1, about 5:1 to about 12:1, about 5:1 to about 10:1, about 5:1 to about 8:1, or about 5:1 to about 7:1, including any combination, sub-combination, range, or sub-range thereof.

(f) Water-Soluble Solvents

The term “water-soluble solvent” is interchangeable with the term “water-miscible solvent” and relates to organic compounds that are liquid at 25° C. and at atmospheric pressure (760 mmHg), and have a solubility of at least 50% in water under these conditions. In some cases, the water-soluble solvent has a solubility of at least 60%, 70%, 80%, or 90%. Non-limiting examples of water-soluble solvents include, for example, glycerin, alcohols (for example, C₁₋₁₅, C₁₋₁₀, or C₁₋₄ alcohols), organic solvents, polyols (polyhydric alcohols), glycols (e.g., butylene glycol, caprylyl glycol, etc.), and a mixture thereof.

Non-limiting examples of water-soluble solvents include monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of are ethylene glycol, propylene glycol, butylene glycol, hexylene glycol, propane diol, and glycerin. The organic solvents can be volatile or non-volatile compounds.

Further non-limiting examples of water-soluble solvents include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

In some instances, polhydric alcohols may be particularly useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In a preferred embodiment, the hair treatment compositions include one or more water-soluble solvents chosen from chosen glycerin, C₁₋₆ mono-alcohols, polyols (polyhydric alcohols), glycols, and a mixture thereof. In an even more preferred embodiment, the hair treatment compositions include one or more water-soluble solvents chosen from glycerin, C₁₋₆ mono-alcohols, and a combination therefore, in particular, a combination of glycerin and a mono-alcohol chosen from isopropanol, ethanol, and mixtures thereof.

The amount of water-soluble solvents in the hair treatment compositions will vary. Nonetheless, in various embodiments, the total amount of the one or more water-soluble solvents is from about 0.1 to about 20 wt. %, based on the total weight of the hair treatment compositions. In certain embodiments, the total amount of the one or more water-soluble solvents is from about 0.1 to about 15 wt. %, about 0.1 to about 12 wt. %, about 0.1 to about 10 wt. %, about 0.1 to about 8 wt. %, about 0.5 to about 20 wt. %, about 0.5 to about 15 wt. %, about 0.5 to about 12 wt. %, about 0.5 to about 10 wt. %, about 0.5 to about 8 wt. %, about 1 to about 20 wt. %, about 1 to about 15 wt. %, about 1 to about 12 wt. %, about 1 to about 10 wt. %, about 1 to about 8 wt. %, about 2 to about 20 wt. %, about 2 to about 15 wt. %, about 2 to about 12 wt. %, about 2 to about 10 wt. %, about 2 to about 8 wt. %, about 4 to about 20 wt. %, about 4 to about 15 wt. %, about 4 to about 12 wt. %, about 4 to about 10 wt. %, or about 4 to about 8 wt. %, including any combination, sub-combination, range, or sub-range thereof by weight, based on the total weight of the hair treatment composition.

(g) Nonionic Thickening Polymers

Non-limiting examples of nonionic thickening polymers include polyvinylpyrrolidone, polyacrylamide, a neutral polysaccharide, and derivatives (such as ethers and esters) thereof. Examples of polysaccharide derivatives include neutral gums (such as guar gum and hydroxypropylguar), cellulose ethers (such as hydroxyethylcellulose (HEC), methylhydroxyethylcellulose (MHEC), ethylhydroxyethylcellulose (EHEC), methylethylhydroxyethylcellulose (MEHEC), hydroxypropylcellulose (HPC), hydroxypropylmethylcellulose (HPMC), and hydrophobized derivatives (e.g., HM-EHEC) thereof), and starch and derivatives (e.g., dextrin) thereof.

Nonlimiting examples of useful nonionic thickening polymers are provided below.

(1) Celluloses modified with groups comprising at least one fatty chain; examples that may be mentioned include: hydroxyethylcelluloses modified with groups comprising at least one fatty chain, such as alkyl, arylalkyl or alkylaryl groups, or mixtures thereof, and in which the alkyl groups are preferably C₈-C₂₂, for instance the product NATROSOL PLUS GRADE 330 CS (C₁₆ alkyls) sold by the company Aqualon, or the product BERMOCOLL EHM 100 sold by the company Berol Nobel; and hydroxyethylcelluloses modified with alkylphenyl polyalkylene glycol ether groups, such as the product AMERCELL POLYMER HM-1500 (polyethylene glycol (15) nonylphenyl ether) sold by the company Amerchol,

(2) Hydroxypropyl guars including hydroxypropyl guars modified with groups comprising at least one fatty chain, such as the product ESAFLOR HM 22 (C₂₂ alkyl chain) sold by the company Lamberti, and the products RE210-18 (C₁₄ alkyl chain) and RE205-1 (C₂₀ alkyl chain) sold by the company Rhone-Poulenc. In a particularly preferred embodiment, the hair treatment compositions include hydroxypropyl guar.

(3) Copolymers of vinylpyrrolidone and of fatty-chain hydrophobic monomers; examples that may be mentioned include: the products ANTARON V216 or GANEX V216 (vinylpyrrolidone/hexadecene copolymer) sold by the company I.S.P. the products ANTARON V220 or GANEX V220 (vinylpyrrolidone/eicosene copolymer) sold by the company I.S.P.,

(4) Copolymers of C₁-C₆ alkyl methacrylates or acrylates and of amphiphilic monomers comprising at least one fatty chain, for instance the oxyethylenated methyl acrylate/stearyl acrylate copolymer sold by the company Goldschmidt under the name ANTIL 208,

(5) Copolymers of hydrophilic methacrylates or acrylates and of hydrophobic monomers comprising at least one fatty chain, for instance the polyethylene glycol methacrylate/lauryl methacrylate copolymer,

(6) Polyurethane polyethers comprising in their chain both hydrophilic blocks usually of polyoxyethylenated nature and hydrophobic blocks, which may be aliphatic sequences alone and/or cycloaliphatic and/or aromatic sequences,

(7) Polymers with an aminoplast ether backbone containing at least one fatty chain, such as the PURE THIX compounds sold by the company Sud-Chemie.

In some instances, the nonionic polymers are chosen from polysaccharides including galactomannan polysaccharide (for example guar derivatives such as hydroxypropyl guar), nonionic derivatives of polysaccharides, associative polymers, and mixtures thereof. In some cases, preferred nonionic polymers include sclerotium gum, guar gums, hydroxyalkyl celluloses optionally modified with a hydrophobic group, such as hydroxyethylcelluloses, hydroxymethylcelluloses optionally modified with a hydrophobic group, guar gum, derivatives of guar gum such as hydroxypropyl guar, and inulins optionally modified with a hydrophobic group.

In a preferred embodiment, the one or more nonionic polymers are chosen from methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, guar gum, hydroxypropyl guar, starch, modified starch, methylhydroxypropyl starch, and a mixture thereof.

Hydroxypropyl guar gum is a particularly preferred nonionic thickening polymer for use in the hair treatment compositions of the instant disclosure.

The total amount of the one or more nonionic thickening polymer will vary depending on the desired thickness and the type of nonionic thickening polymer used. Nonetheless, in various embodiments, the hair treatment compositions include about 0.05 to about 5 wt. % of the one or more nonionic polymers, based on the total weight of the hair treatment composition. IN further embodiments, the total amount of the one or more nonionic thickening agents is from about 0.05 to about 4 wt. %, about 0.05 to about 3 wt. %, about 0.05 to about 2 wt. %, about 0.05 to about 1.5 wt. %, about 0.05 to about 1 wt. %, about 0.1 to about 5 wt. %, about 0.1 to about 4 wt. %, about 0.1 to about 3 wt. %, about 0.1 to about 2 wt. %, about 0.1 to about 1.5 wt. %, about 0.1 to about 1 wt. %, about 0.2 to about 5 wt. %, about 0.2 to about 4 wt. %, about 0.2 to about 3 wt. %, about 0.2 to about 3 wt. %, about 0.2 to about 2 wt. %, or about 0.2 to about 1.5 wt. %, or about 0.2 to about 1 wt. %, including any combination, sub-combination, range, or sub-range thereof by weight, based on the total weight of the hair treatment composition.

(h) Conditioning Agents

Nonlimiting examples of conditioning agents include cationic polymers, silicones, humectants, emollients, occlusives, proteins, etc. A variety of useful conditioning agents are described in the INTERNATIONAL COSMETIC INGREDIENT DICTIONARY AND HANDBOOK (Volume 4, publisher: R C Pepe, J A Wenninger, G N McEwen, The Cosmetic, Dental and Fragrance Association, 9th edition, 2002) under Section 4 under the heading “Hair Conditioning Agents,” which is incorporated herein by reference in its entirety. Particularly preferred conditioning agents include cationic polymers, silicones, and mixtures thereof. Accordingly, in a particularly preferred embodiment, the hair treatment compositions of the instant disclosure include one or more cationic polymers, silicones, or a mixture thereof; and optionally, one or more additional conditioning agents.

The total amount of the one or more conditioning agents in the hair treatment compositions will vary depending on the type of conditioning agent used. For example, cationic polymers are typically used in lower amounts than silicones, i.e., generally a higher amount of silicones are used than cationic polymers. Nonetheless, in various embodiments, the total amount of the one or more conditioning agents is from about 0.05 to about 15 wt. %, based on the total weight of the hair treatment composition. In further embodiments, the total amount of the one or more conditioning agents is rom about 0.05 to about 12 wt. %, about 0.05 to about 10 wt. %, about 0.05 to about 8 wt. %, about 0.1 to about 15 wt. %, about 0.1 to about 12 wt. %, about 0.1 to about 10 wt. %, about 0.1 to about 8 wt. %, including any combination, sub-combination, range, or sub-range thereof by weight, based on the total weight of the hair treatment composition. Amounts more specific to cationic polymers and to silicones are provided in the sections below.

(h-1) Cationic Polymers

Cationic polymers for purposes of the instant disclosure are polymers bearing a positive charge or incorporating cationic entities in their structure. The cationic polymers can comprise mixtures of monomer units derived from amine- and/or quaternary ammonium-substituted monomer and/or compatible spacer monomers. Cationic polymers often provide conditioning benefits to the hair treatment compositions and therefore may be referred to as “cationic conditioning polymers.” Non-limiting examples of cationic polymers include copolymers of 1-vinyl-2-pyrrolidine and 1-vinyl-3-methyl-imidazolium salt (e.g., chloride salt) (referred to as Polyquaternium-16); copolymers of 1-vinyl-2-pyrrolidine and dimethylaminoethyl methacrylate (referred to as

Polyquaternium-11); cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7); polysaccharide polymers, such as cationic cellulose derivatives and cationic starch derivatives. Cationic cellulose is available as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). Additionally or alternatively, the cationic conditioning polymers may include or be chosen from cationic guar gum derivatives, such as guar hydroxypropyltrimonium chloride.

Preferred cationic polymers include cationic polysaccharide polymers, such as cationic cellulose, cationic starch, and cationic guar gum. In the context of the instant disclosure cationic polysaccharide polymers include cationic polysaccharides and polysaccharide derivatives (e.g., derivatized to be cationic), for example, resulting in cationic cellulose (cellulose derivatized to be cationic), cationic starch (derivatized to be cationic), cationic guar (guar derivatized to be cationic).

Non-limiting examples of cationic celluloses include hydroxyethylcellulose (also known as HEC), hydroxymethylcellulose, methylhydroxyethylcellulose, hydroxypropylcellulose (also known as HPC), hydroxybutylcellulose, hydroxyethylmethylcellulose (also known as methyl hydroxyethylcellulose) and hydroxypropylmethylcellulose (also known as HPMC), cetyl hydroxyethylcellulose, polyquaternium-10, polyquaternium-24, and mixtures thereof, preferably polyquaternium-10, polyquaternium-24, and mixtures thereof.

Non-limiting examples of cationic guar include guar hydroxypropyltrimonium chloride, hydroxypropyl guar hydroxypropyltrimonium chloride, guar hydroxypropyltrimethylammonium chloride, and mixtures thereof.

Non-limiting examples of cationic starch include starch hydroxypropyltrimonium chloride, hydroxypropyl oxidized starch PG trimonium chloride, and a mixture thereof.

In certain embodiments, the hair treatment composition may include one or more polyquaterniums. Nonlimiting examples include polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof.

In certain embodiments, the hair treatment composition may include polyquaternium-1 (ethanol, 2,2′,2″ -nitrilotris-, polymer with 1,4-dichloro-2-butene and N,N,N′,N′-tetramethyl-2-butene-1,4-diamine), polyquaternium-2, (poly[bis(2-chloroethyl) ether-alt-1,3-bis[3-(dimethylamino)propyl]urea]), polyquaternium-4, (hydroxyethyl cellulose dimethyl diallylammonium chloride copolymer; Diallyldimethylammonium chloride-hydroxyethyl cellulose copolymer), polyquaternium-5 (copolymer of acrylamide and quaternized dimethylammoniumethyl methacrylate), polyquaternium-6 (poly(diallyldimethylammonium chloride)), polyquaternium-7 (copolymer of acrylamide and diallyldimethylammonium chloride), polyquaternium-8 (copolymer of methyl and stearyl dimethylaminoethyl ester of methacrylic acid, quaternized with dimethylsulphate), polyquaternium-9 (homopolymer of N,N-(dimethylamino)ethyl ester of methacrylic acid, quaternized with bromomethane), polyquaternium-10 (quaternized hydroxyethyl cellulose), polyquaternium-11 (copolymer of vinylpyrrolidone and quaternized dimethylaminoethyl methacrylate), polyquaternium-12 (ethyl methacrylate/abietyl methacrylate/diethylaminoethyl methacrylate copolymer quaternized with dimethyl sulfate), polyquaternium-13 (ethyl methacrylate/oleyl methacrylate/diethylaminoethyl methacrylate copolymer quaternized with dimethyl sulfate), polyquaternium-14 (trimethylaminoethylmethacrylate homopolymer), polyquaternium-15 (acrylamide-dimethylaminoethyl methacrylate methyl chloride copolymer), Polyquaternium-16 (copolymer of vinylpyrrolidone and quaternized vinylimidazole),

Polyquaternium-17 (adipic acid, dimethylaminopropylamine and dichloroethylether copolymer), Polyquaternium-18 (azelanic acid, dimethylaminopropylamine and dichloroethylether copolymer), polyquaternium-19 (copolymer of polyvinyl alcohol and 2,3-epoxypropylamine), polyquaternium-20 (copolymer of polyvinyl octadecyl ether and 2,3-epoxypropylamine), polyquaternium-22 (copolymer of acrylic acid and diallyldimethylammonium chloride), polyquaternium-24 (auaternary ammonium salt of hydroxyethyl cellulose reacted with a lauryl dimethyl ammonium substituted epoxide), polyquaternium-27 (block copolymer of Polyquaternium-2 and Polyquaternium-17), polyquaternium-28 (copolymer of vinylpyrrolidone and methacrylamidopropyl trimethylammonium), polyquaternium-29 (chitosan modified with propylen oxide and quaternized with epichlorhydrin), polyquaternium-30 (ethanaminium, N-(carboxymethyl)-N,N-dimethyl-2-[(2-methyl-1-oxo-2-propen-1-yl)oxy]-, inner salt, polymer with methyl 2-methyl-2-propenoate), polyquaternium-31 (N,N- dimethylaminopropyl-N-acrylamidine quatemized with diethylsulfate bound to a block of polyacrylonitrile), polyquaternium-32 (poly(acrylamide 2-methacryloxyethyltrimethyl ammonium chloride)), polyquaternium-33 (copolymer of trimethylaminoethylacrylate salt and acrylamide), polyquaternium-34 (copolymer of 1,3-dibromopropane and N,N-diethyl-N′,N′-dimethyl-1 ,3-propanediamine), Polyquaternium-35 (methosulphate of the copolymer of methacryloyloxyethyltrimethylammonium and of methacryloyloxyethyldimethylacetylammonium), polyquaternium-36 (copolymer of N,N-dimethylaminoethylmethacrylate and buthylmethacrylate, quaternized with dimethylsulphate), polyquaternium-37 (poly(2-methacryloxyethyltrimethylammonium chloride)), polyquaternium-39 (terpolymer of acrylic acid, acrylamide and diallyldimethylammonium Chloride), polyquaternium-42 (poly[oxyethylene(dimethylimino)ethylene (dimethylimino)ethylene dichloride]), olyquaternium-43 (copolymer of acrylamide, acrylamidopropyltrimonium chloride, 2-amidopropylacrylamide sulfonate and dimethylaminopropylamine), polyquaternium-44 (3-Methyl-1-vinylimidazolium methyl sulfate-N-vinylpyrrolidone copolymer), polyquaternium-45 (copolymer of (N-methyl-N-ethoxyglycine)methacrylate and N,N-dimethylaminoethylmethacrylate, quaternized with dimethyl sulphate), polyquaternium-46 (terpolymer of vinylcaprolactam, vinylpyrrolidone, and quaternized vinylimidazole), polyquaternium-47 (terpolymer of acrylic acid, methacrylamidopropyl trimethylammonium chloride, and methyl acrylate), and/or polyquaternium-67.

In certain embodiments, the hair treatment compositions of the instant disclosure include one or more cationic polymers selected from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful. A particularly preferred and useful cationic polymer is polyquaternium-10.

In certain embodiments, the hair treatment compositions include one or more cationic polymers chosen from cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, and mixtures thereof.

The hair treatment compositions according to the instant disclosure typically include about 0.1 to about 5 wt. % of one or more cationic surfactants, based on the total weight of the hair treatment composition. The hair treatment compositions may include about 0.1 to about 4 wt. %, about 0.1 to about 3 wt. %, about 0.1 to about 2 wt. %, about 0.1 to about 1.5 wt. %, about 0.2 to about 5 wt. %, about 0.2 to about 4 wt. %, about 0.2 to about 3 wt. %, about 0.2 to about 2 wt. %, about 0.2 to about 1.5 wt. % of the one or more cationic polymers, including any combination, sub-combination, range, or sub-range thereof by weight, based on the total weight of the hair treatment composition.

(h-2) Silicones

Silicones are also referred to as silicone oils. Nonlimiting examples of silicones include dimethicone, dimethiconol, dimethiconol, cyclomethicone, polysilicone-11, phenyl trimethicone, trimethylsilylamodimethicone, and stearoxytrimethylsilane. In a preferred embodiment, the one or more silicones are chosen from non-volatile silicon oils. Useful silicone oils include polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates. Other examples of silicone oils that may be mentioned include volatile linear or cyclic silicones, such as those with a viscosity 8 centistokes (8x106 m2/s) and/or containing from 2 to 7 silicon atoms. These silicones optionally comprise alkyl or alkoxy groups containing from 1 to 10 carbon atoms. Non-limiting examples of volatile silicone oils include octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, or mixtures thereof.

In a preferred embodiment, the hair treatment compositions include one or more silicone oils chosen from dimethicone, dimethiconol, cyclomethicone, polysilicone-11, phenyl trimethicone, amodimethicone, and quaternized or quaternizable polysiloxanes (for example, bis-cetearyl amodimethicone), and mixtures thereof.

Nonlimiting examples of quaternized or quaternizable polysiloxanes include silicone quaternium 22 (e.g., ABIL T QUAT 60), silicone quaternium 12 (PECOSIL CA-1240), amodimethicone (e.g., DOW CORNING 2-8566 AMINO FLUID), bis-cetearyl amodimethicone (e.g., SILSOFT AX), bis-amino PEG/PPG-41/3 aminoethyl PG-propyl dimethicone (e.g., SILSOFT A-843), PEG-40/PPG-8 methylaminopropyl hydroxypropyl dimethicone copolymer (e.g., SILSOFT A+), silicone quaterium 16 (and) undeceth-11 (and) butyloctanol (and) undeceth-5 (e.g., DOW CORNING 5-7113 SILICONE QUAT MICROEMULSION), and bis-isobutyl/PEG/PPG-20/35/amodimethicone copolymer (e.g., DOW CORNING CE 8401 EMULSION). A preferred quaternized or quaternizable polysiloxane is bis-cetearyl amodimethicone.

In certain embodiments, at least one of the one or more silicones (at least one of the one or more conditioning agents) is an amino functionalized silicone. Nonlimiting examples include amodimethicone, bis-hydroxy/methoxy amodimethicones, bis-cetearyl amodimethicone, amodimethicone, bis(C₁₃-15 alkoxy) PG amodimethicones, aminopropyl phenyl trimethicones, aminopropyl dimethicones, bis-amino PEG/PPG-41/3 aminoethyl PG-propyl dimethicones, caprylyl methicones, and a mixture thereof. Amodimethicone is a particularly preferred amino functionalized silicone.

In a certain embodiments at least one or the one or more silicone oils are chosen from dimethicone, dimethiconol, cyclopentasiloxane, cyclomethicone, cyclotetrasiloxane, cyclohexasiloxane, cycloheptasiloxane, decamethylcyclopentasiloxane, cyclotetrasiloxane, cyclotrisiloxane, capryldimethicone, caprylyl trimethicone, caprylyl methicone, cetearylmethicone, hexadecylmethicone, hexylmethicone, lauryl methicone, myristyl methicone, phenyl methicone, stearyl methicone, stearyl dimethicone, behenyl dimethicone, trifluoropropyl methicone, cetyl dimethicone, polyphenylmethylsiloxane, dimethylpolysiloxane, methylphenylpolysiloxane, methyltrimethicone, diphenylsiloxyphenyl trimethicone, and phenyl trimethicone, and mixtures thereof, preferably dimethicone. In a preferred embodiment, the hair treatment compositions include dimethicone and optionally, one or more additional silicones, for example, one or more additional silicones chosen from amodimethicone, quaternized or quaternizable amodimethicone (e.g., bis-cetearyl amodimethicone), or a combination thereof.

The total amount of the one or more silicones in the hair treatment compositions, if present, will vary. Nonetheless, in various embodiments, the total amount of the one or more silicones is from about 0.1 to about 15 wt. %, based on the total weight of the hair treatment composition. In further embodiments, the total amount of the one or more silicones is from about 0.1 to about 12 wt. %, about 0.1 to about 10 wt. %, about 0.1 to about 8 wt. %, about 1 to about 15 wt. %, about 1 to about 12 wt. %, about 1 to about 10 wt. %, about 1 to about 8 wt. %, about 2 to about 15 wt. %, about 2 to about 12 wt. %, about 2 to about 10 wt. %, about 2 to about 8 wt. %, about 3 to about 15 wt. %, about 3 to about 12 wt. %, about 3 to about 10 wt. %, or about 3 to about 8 wt. %, including any combination, sub-combination, range, or sub-range thereof by weight, based on the total weight of the hair treatment composition.

(i) Miscellaneous Ingredients

In various embodiments, the hair treatment compositions of the instant disclosure include one or more miscellaneous ingredients. Miscellaneous ingredients are ingredients that are compatible with the hair treatment compositions and do not disrupt or materially affect the basic and novel properties of the hair treatment compositions. Miscellaneous ingredients commonly used in cosmetics and hair care products are known in the art. Non-limiting examples include preservatives, fragrances, pH adjusters, salts, antioxidants, vitamins, vitamin derivatives, botanical extracts, UV filtering agents, proteins, protein hydrolysates, protein isolates, hydrotropes, pearlescent agents, buffers, colorants for coloring the hair treatment compositions (not for coloring the hair), sequestering agents, etc.

The total amount of the one or more miscellaneous ingredients, if present, will vary. Nonetheless, in various embodiments, the total amount of the one or more miscellaneous ingredients is from about 0.01 to about 10 wt. %, based on the total weight of the hair treatment composition. In further embodiments, the total amount of the one or more miscellaneous ingredients is from about 0.01 to about 8 wt. %, about 0.01 to about 5 wt. %, about 0.01 to about 3 wt. %, about 0.1 to about 10 wt. %, about 0.1 to about 8 wt. %, about 0.1 to about 5 wt. %, about 0.1 to about 3 wt. %, about 1 to about 10 wt. %, about 1 to about 8 wt. %, about 1 to about 5 wt. %, or about 1 to about 3 wt. %, including any combination, sub-combination, range, or sub-range thereof by weight, based on the total weight of the hair treatment composition.

(k) pH

The pH of the hair treatment compositions will vary. For example, the pH of the hair treatment composition can vary depending upon how the compositions is formulated for use. For example, when the hair treatment compositions is formulated as a stand-alone product, the pH may differ from when the hair treatment composition is formulated for addition or use with another hair treatment composition (for example, a hair treatment composition comprising one or more chemically active agents) or when the hair treatment composition is formulated for use in a routine (for example, for use in a shampoo and conditioning routine). Nonetheless, in various embodiments, the pH of the hair treatment composition is from about 3 to about 10. In certain embodiments, the pH of the hair treatment compositions is from about 4 to about 10, about 5 to about 10, about 6 to about 10, or about 7 to about 10. In additional embodiments, the pH of the hair treatment compositions is from about 3 to about 9, about 3 to about 9, about 3 to about 8, about 3 to about 7, preferably from about 3 to about 6.

Viscosity of the Hair Treatment Compositions

The viscosity of the hair treatment compositions is typically about 5 mPa·s to about 100,000 mPa·s, about 5 mPa·s to about 75,000 mPa·s, about 5 mPa·s to about 50,000 mPa·s at a temperature of 25° C. The viscosity may be about 10 mPa·s to about 100,000 mPa·s, about 10 mPa·s to about 75,000 mPa·s , about 10 mPa·s to about 50,000 m Pas, about 15 to about 50,000 mPa·s, about 20 to about 50,000 mPa·s, about 100 m Pas to about 50,000 m Pas, about 1,000 to about 50,000, about 5 mPa·s to about 25,000, about 10 to about 25,000 mPa·s, about 20 to about 25,000 mPa·s, about 100 to about 25,000 m Pas, about 1,000 to about 25,000 mPa·s, about 5 to about 10,000 mPa·s, about 15 to about 10,000 mPa·s, about 20 mPa·s to 10,000 mPa·s, about 5 mPa·s to 50,000 mPa·s, about 10 mPa·s to 50,000 mPa·s, about 15 mPa·s to 50,000 mPa·s, about 20 mPa·s to 50,000 mPa·s, about 5 to 25,000 mPa·s, about 10 mPa·s to 25,000 mPa·s, about 15 to 25,000 mPa·s, about 20 mPa·s to 25,000 mPa·s, about 5 mPa·s to 10,000 mPa·s, about 10 mPa·s to 10,000 mPa·s, about 15 mPa·s to 10,000 mPa·s, or about 20 to 10,000 mPa·s, about 5 mPa·s to about 5,000 mPa·s, about 10 mPa·s to about 5,000 mPa·s, about 15 mPa·s to about 5,000 mPa·s, about 20 mPa·s to about 5,000 mps, about 5 mPa·s to about 1,000 mPa·s, about 10 m Pas to about 1,000 mPa·s, about 15 mPa·s to about 1,000 mPa·s, about 20 to about 1,000 mPa·s, about 5 to about 500 mPa·s, about 10 to about 500 mPa·s, about 15 to about 500 mPa·s, about 20 to about 500 mPa·s, or about 5 to about 250 mPa·s at a temperature of 25° C., including any combination, sub-combination, range, or sub-range thereof.

The viscosity measurements can be carried out, for example, using a Br000ksfield viscometer/rheometer using a RV-3 Disk or an RV-5 Disk spindle at a particular speed, for example, 5, 10, 15, and/or 20 rpm or using a Rheomat with an M4 spindle. For example, viscosity can be measured with T-bar E spindle using a Helipath stand mounted on Brookfield DV-II+Pro, with a rotational speed of 20 RPM.

Stability

The hair treatment compositions are stable and homogenous. In other words, the hair treatment compositions do not visually phase separate or develop sedimentation, and do not form visibly observable particulates. For instance, the hair treatment composition remain stable for at least 1 week at room temperature; the hair treatment composition remain stable for at least 8 weeks at 4° C.; and/or the hair treatment composition remain stable for at least 8 weeks at 45° C. Accordingly, the instant disclosure relates to hair treatment compositions that remain visually homogenous and free from visual phase separation and particulate formation for at least 1 week, 4 weeks, and/or 8 weeks at room temperature. Furthermore, the instant disclosure relates to hair treatment compositions that remain visually homogenous and free from visual phase separation and particulate formation for at least 1 week, 4 weeks, and/or 8 weeks at 4° C. The instant disclosure also relates to hair treatment compositions that remain visually homogenous and free from visual phase separation and particulate formation for at least 1 week, 4 weeks, and/or 8 weeks at 45° C.

Methods

The hair treatment compositions are particularly useful in methods for treating hair, for example methods for restructuring, repairing, strengthening, and/or protecting hair. The methods repair, minimize, mitigate, and/or compensate for damage caused to hair by chemical treatments (e.g., bleaching, coloring, straightening, curling, etc.). The methods also relate to preventing, minimizing, mitigating, and treating hair vulnerable to damage (or already damaged) by environmental factors (e.g., hard water, sun damage, chlorine, etc.) and to damage caused by styling the hair, for example, with heat (e.g., blow drying, hot irons, etc.).

The methods include applying one or more hair treatment compositions of the instant disclosure to the hair. The hair treatment composition(s) may be temporarily applied to the hair (e.g., applied for a period of about 1 hour or less and rinsed from the hair), or may be applied to the hair and allowed to remain on the hair for a longer period of time before being rinsed from the hair (e.g., for about 2 minutes, 5 minutes, 10 minutes, 20 minutes, 30 minutes, or longer). In situations where the hair treatment composition is rinsed from the hair shortly after application (immediately after application or within about 2, 5, 10, 20, 30 minutes or longer) before styling the hair, the hair treatment compositions is a “rinse off” product. In certain embodiments, the hair treatment compositions are applied to the hair and allowed to remain on the hair indefinitely, for example, allowed to remain on the hair without rinsing from the hair before styling the hair, i.e., the hair treatment composition is a “leave-on” product. The methods are particularly well suited for application to chemically treated hair, hair to be subjected to a chemical treatment, damaged hair, and natural hair, for example, to improve the hair's strength, luster, shine, and/or elasticity, etc.

The hair treatment compositions of the instant case are particularly advantageous because they can be a stand-alone product (used independently) or can be used in methods that combine the hair treatment compositions of the instant disclosure with an independent hair treatment composition for chemically treating hair (i.e., a hair treatment composition comprising one or more chemically active agents that chemically treat the hair). Nonlimiting examples of hair treatment compositions for chemically treating hair include bleaching compositions, hair straightening or curling compositions, hair relaxing or texlaxing compositions, and the like. The hair treatment compositions of the instant case may be combined with (added into) one or more components of a hair treatment composition for chemically treating hair. For example, if the hair treatment composition for chemically treating hair is a bleaching composition, the hair treatment compositions of the instant case may be added into a developer component, may be added into a bleaching component, or may be combined with a mixture of the developer component and the bleaching component. Similarly, hair shaping compositions (for perming, straightening, and/or relaxing hair) often require more than one component (e.g., a relaxing component, an activator component, a neutralizer component, etc.). The hair treatment compositions of the instant disclosure may be included in one or more (or all) of the components.

Similar to the above, the hair treatment compositions of the instant disclosure may be used as a stand-alone product in a hair cleansing (shampooing) and optional conditioning routine, or may be combined with (added into) a hair cleanser (shampoo), conditioner, or added into both a hair cleanser and conditioner.

The instant disclosure relates methods for making the hair treatment compositions. The methods include combining: (a) both citric acid and salts of citric acid in a weight ratio of from 1:10 to about 10:1 (or according to any of the ratios described throughout the disclosure); (b) one or more surfactants (according to the amounts and types described throughout the disclosure); and water (for example, according to the amounts described throughout the disclosure; to form a hair treatment composition according to the instant disclosure. The citric acid and the salts of citric acid can be combined with each other before being added to the other components of the hair treatment composition or can be individual added/combined with the other components of the hair treatment composition.

In some instanced, to obtain a desired pH, the methods may include adding one or more pH adjusting compounds, for example, to ensure a pH of from about 3 to about 10 (or any other pH value or range described throughout the instant disclosure.

The methods also optionally include combining (d) one or more fatty alcohols (according to the amounts and types described throughout the disclosure); (e) one or more fatty compounds other than fatty alcohols(according to the amounts and types described throughout the disclosure); (f) one or more water-soluble solvents (according to the amounts and types described throughout the disclosure); (g) one or more nonionic thickening polymers (according to the amounts and types described throughout the disclosure); (h) one or more conditioning agents chosen from cationic polymers, silicones, and mixtures thereof (according to the amounts and types described throughout the disclosure); and/or (i) one or more miscellaneous ingredients (according to the amounts and types described throughout the disclosure).

Kits

The hair treatment compositions of the instant disclosure, in various embodiments, are part of a kit, for example, a kit comprising one or more hair treatment compositions according to the instant disclosure and one or more additional hair treatment compositions, wherein each of the one or more hair treatment compositions (and each of the additional hair treatment compositions) are separately contained. For instance, in certain embodiments, the kits comprise: (i) one or more hair treatment compositions according to the instant disclosure; and (ii) one or more additional hair treatment compositions chosen from hair cleansing compositions (shampoos), hair conditioning compositions (conditioners), and hair treatment compositions for chemically treating hair comprising one or more chemically active agents that chemically treat the hair.

### Embodiments

Preferred embodiments are discussed throughout the instant disclosure and below. In various embodiments, the hair treatment compositions comprise or consists of:
(a) about 0.25 to about 10 wt. %, preferably about 0.25 to about 6, more preferably about 0.25 to about 3 wt. % of citric acid, one or more salts of citric acid, or a combination thereof, wherein if a combination of citric acid and salts of citric acid is present, the weight ratio of citric acid to salts of citric acid added to form the combination is from 1:10 to about 10:1,
   alternatively, however, the hair treatment composition comprises about 1 to about 10 wt. %, preferably, about 1.2 to about 8 wt. %, more preferably about 1.5 to about 5 wt. % of a combination of citric acid and one or more salts of citric acid;
   wherein the weight ratio of citric acid to salts of citric acid added to form the combination is from 1:10 to about 10:1, preferably from about 1:6 to about 6:1, more preferably from about 1:3 to about 3:1
(b) one or more surfactants, preferably one or more cationic surfactants and optionally, one or more surfactants chosen from nonionic surfactants, amphoteric surfactants, and a mixture thereof;
(c) about 50 to about 95 wt. %, preferably about 55 to about 90 wt. %, more preferably about 60 to about 85 wt. % of water;
(d) optionally, about 0.1 to about 10 wt. %, preferably about 0.5 to about 8 wt. %, more preferably about 1 to about 6 wt. %, of one or more miscellaneous ingredients, for example, chosen from preservatives, fragrances, pH adjusters, salts, antioxidants, vitamins, vitamin derivatives, botanical extracts, UV filtering agents, proteins, protein hydrolysates, protein isolates, hydrotropes, pearlescent agents, buffers, colorants for coloring the hair treatment compositions (not for coloring the hair), sequestering agents, and a combination thereof;
   wherein the pH of the composition is from about 3 to about 10, preferably from about 4 to about 9, more preferably from about 5 to about 8; and weight percentages are based on the total weight of the composition.

In certain embodiments, the hair treatment composition comprises or consists of:
(a) about 0.25 to about 10 wt. %, preferably about 0.25 to about 6, more preferably about 0.25 to about 3 wt. % of citric acid, one or more salts of citric acid, or a combination thereof, wherein if a combination of citric acid and salts of citric acid is present, the weight ratio of citric acid to salts of citric acid added to form the combination is from 1:10 to about 10:1,
   alternatively, however, the hair treatment composition comprises about 1 to about 10 wt. %, preferably, about 1.2 to about 8 wt. %, more preferably about 1.5 to about 5 wt. % of a combination of citric acid and one or more salts of citric acid;
   wherein the weight ratio of citric acid to salts of citric acid added to form the combination is from 1:10 to about 10:1, preferably from about 1:6 to about 6:1, more preferably from about 1:3 to about 3:1;
(b) one or more surfactants, preferably one or more cationic surfactants and optionally, one or more surfactants chosen from nonionic surfactants, amphoteric surfactants, and a mixture thereof;
(c) about 50 to about 95 wt. %, preferably about 55 to about 90 wt. %, more preferably about 60 to about 85 wt. % of water;
(d) one or more fatty alcohols;
(e) one or more fatty compounds other than fatty alcohols, for example, chosen from oils, waxes, alkanes (paraffins), fatty acids, fatty esters, triglyceride compounds, lanolin, derivatives thereof, and mixtures thereof;
   wherein the weight ratio of (d) to (e) is from about 2:1 to about 15:1, preferably from about 3:1 to about 10:1, more preferably from about 4:1 to about 8:1.
(f) one or more water-soluble solvents, for example, one or more water-soluble solvents chosen from glycerin, C₁₋₆ mono-alcohols, polyols (polyhydric alcohols), glycols, and a mixture thereof;
(g) one or more nonionic thickening polymers, for example, chosen from guar gum, guar derivatives (e.g., hydroxypropyl guar), cellulose gum, cellulose derivatives, starch, starch derivatives, polysaccharides, polysaccharide derivatives, homopolymers and copolymers of ethylene oxide having a molar mass equal to or greater than 10,000 g/mol, polyvinyl alcohols, homopolymers and copolymers of vinylpyrrolidone, homopolymers and copolymers of vinylcaprolactam, homopolymers and copolymers of polyvinyl methyl ether, and mixtures thereof, more preferably, the one or more nonionic thickening polymers includes hydroxypropyl guar;
(h) optionally, one or more conditioning agents, preferably chosen from cationic polymers, silicones, and a combination thereof;
(i) optionally, about 0.1 to about 10 wt. %, preferably about 0.5 to about 8 wt. %, more preferably about 1 to about 6 wt. %, of one or more miscellaneous ingredients, for example, chosen from preservatives, fragrances, pH adjusters, salts, antioxidants, vitamins, vitamin derivatives, botanical extracts, UV filtering agents, proteins, protein hydrolysates, protein isolates, hydrotropes, pearlescent agents, buffers, colorants for coloring the hair treatment compositions (not for coloring the hair), sequestering agents, and a combination thereof;
   wherein the pH of the composition is from about 3 to about 10, preferably from about 4 to about 9, more preferably from about 5 to about 8; and weight percentages are based on the total weight of the composition.

The hair treatment composition is preferably in the form of an oil-in-water emulsion.

In certain embodiments, the hair treatment composition comprises or consists of:
(a) about 0.25 to about 10 wt. %, preferably about 0.25 to about 6, more preferably about 0.25 to about 3 wt. % of citric acid, one or more salts of citric acid, or a combination thereof, wherein if a combination of citric acid and salts of citric acid is present, the weight ratio of citric acid to salts of citric acid added to form the combination is from 1:10 to about 10:1,
   alternatively, however, the hair treatment composition comprises about 1 to about 10 wt. %, preferably, about 1.2 to about 8 wt. %, more preferably about 1.5 to about 5 wt. % of a combination of citric acid and one or more salts of citric acid;
   wherein the weight ratio of citric acid to salts of citric acid added to form the combination is from 1:10 to about 10:1, preferably from about 1:6 to about 6:1, more preferably from about 1:3 to about 3:1;
(b) about 1 to about 10 wt. %, preferably about 2 to about 8 wt. %, more preferably 4 to about 8 wt. % of one or more surfactants, preferably one or more cationic surfactants and optionally, one or more surfactants chosen from nonionic surfactants, amphoteric surfactants, and a mixture thereof, more preferably wherein the total amount of cationic surfactants is from about 1 to about 8 wt. %, preferably from about 2 to about 8 wt. %, more preferably from about 4 to about 6 wt. %, based on the total weight of the hair treatment composition,
   wherein the one or more cationic surfactants are preferably chosen from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof, and more preferably are chosen from cetrimonium chloride, behentrimonium chloride, dicetyldimonium chloride, and a mixture thereof;
(c) about 50 to about 95 wt. %, preferably about 55 to about 90 wt. %, more preferably about 60 to about 85 wt. % of water;
(d) about 5 to about 20 wt. %, preferably about 5 to about 15 wt. %, more preferably about 8 to about 15 wt. % of one or more fatty alcohols, for example, one or more fatty alcohols chosen from cetyl alcohol, stearyl alcohol, cetearyl alcohol, behenyl alcohol, lauryl alcohol, myristic or myristyl alcohol, arachidyl alcohol, lignoceryl alcohol, or mixtures thereof;
(e) about 1 to about 10 wt. %, preferably about 1 to about 6 wt. %, more preferably about 1.5 to about 5 wt. % of one or more fatty compounds other than fatty alcohols, for example, chosen from oils, waxes, alkanes (paraffins), fatty acids, fatty esters, triglyceride compounds, lanolin, derivatives thereof, and mixtures thereof;
   wherein the weight ratio of (d) to (e) is from about 2:1 to about 15:1, preferably from about 3:1 to about 10:1, more preferably from about 4:1 to about 8:1.
(f) about 0.5 to about 10 wt. %, preferably about 2 to about 10 wt. %, more preferably about 2 to about 8 wt. % of one or more water-soluble solvents, for example, one or more water-soluble solvents chosen from glycerin, C₁₋₆ mono-alcohols, polyols (polyhydric alcohols), glycols, and a mixture thereof, wherein the one or more water-soluble solvents preferably include at least glycerin, one or more C₁₋₆ mono-alcohols, or a mixture thereof;
(g) about 0.1 to about 5 wt. %, preferably about 0.1 to about 4 wt. %, more preferably about 0.2 to about 3 wt. % of one or more nonionic thickening polymers, preferably chosen from guar gum, guar derivatives (e.g., hydroxypropyl guar), cellulose gum, cellulose derivatives, starch, starch derivatives, polysaccharides, polysaccharide derivatives, homopolymers and copolymers of ethylene oxide having a molar mass equal to or greater than 10,000 g/mol, polyvinyl alcohols, homopolymers and copolymers of vinylpyrrolidone, homopolymers and copolymers of vinylcaprolactam, homopolymers and copolymers of polyvinyl methyl ether, and mixtures thereof, more preferably, the one or more nonionic thickening polymers includes hydroxypropyl guar;
(h) optionally, about 0.01 to about 12 wt. %, preferably about 0.1 to about 10 wt. %, more preferably about 0.1 to about 8 wt. % of one or more conditioning agents, preferably chosen from cationic polymers, silicones, and a combination thereof;
(i) optionally, about 0.1 to about 10 wt. %, preferably about 0.5 to about 8 wt. %, more preferably about 1 to about 6 wt. %, of one or more miscellaneous ingredients, for example, chosen from preservatives, fragrances, pH adjusters, salts, antioxidants, vitamins, vitamin derivatives, botanical extracts, UV filtering agents, proteins, protein hydrolysates, protein isolates, hydrotropes, pearlescent agents, buffers, colorants for coloring the hair treatment compositions (not for coloring the hair), sequestering agents, and a combination thereof;
   wherein the pH of the composition is from about 3 to about 10, preferably from about 4 to about 9, more preferably from about 5 to about 8; and weight percentages are based on the total weight of the composition.

The hair treatment composition is preferably in the form of an oil-in-water emulsion.

In certain embodiments, the hair treatment compositions described throughout the instant case may optionally include one or more amines, e.g., monoamines, diamines, polyamines, and mixtures thereof. Nonetheless, in other embodiments, it is preferrable that the hair treatment compositions are free or essentially free from amines, i.e., free or essentially free from monoamines, diamines, and/or polyamines. In other embodiments, the hair treatment compositions of the instant disclosure are free or essentially free from monoethanolamine. In further embodiments, the hair treatment compositions of the instant disclosure are free or essentially free from monoethanolamine, propanolamine, isopropanolamine, triethanolamine, butanolamine, isobutanolamine, methylethanolamine, butylethanolamine, and mixtures thereof.

In certain embodiments, hair treatment compositions described throughout the instant disclosure may optionally include one or more polymeric acid compounds and/or polymeric acid anhydride compounds, for example, polymers containing recurring units derived from maleic acid and/or maleic anhydride. In certain embodiments, the hair treatment compositions of the instant disclosure are free or essentially free from polymeric acid compounds and/or polymeric acid anhydride compounds are polymers containing recurring units derived from one or more of the following acids, anhydrides, salts, or derivatives thereof:

(i) styrene sulfonic acid,

(ii) crotonic acid,
(iii) acrylic or methacrylic acid,

(iv) ethylene-α,β-dicarboxylic acid, and/or

(v) allyloxyacetic acid, methallyloxyacetic acid, 3-allyloxypropionic acid, allylthioacetic acid, allylaminoacetic acid, vinylacetic acid, vinyloxyacetic acid, crotyloxyacetic acid, 3-butenoic acid, 4-pentenoic acid, 10-undecenoic acid, allylmalonic acid, maleamic acid, itaconamic acid or N-monohydroxyalkyl- or N-dihydroxy-alkyl-maleamic or -itaconamic acids.

As explained below under the heading “Definitions,” when the hair treatment compositions are “essentially free” from a designated component (e.g., one or more amines, one or more polymeric acid compounds and/or polymeric acid anhydride compounds, etc.) there may be less than 2% by weight of the designated component.

For instance, there may be less than 2% by weight of the designated component added to a composition, based on the total weight of the compositions (provided that an amount of less than 2% by weight does not materially affect the basic and novel characteristics of the claimed invention). Similarly, the compositions may include less than 1.5 wt %, less than 1 wt %, less than 0.5 wt %, less than 0.1 wt %, less than 0.05 wt %, or less than 0.01 wt %, or none of the designated component.

### EXAMPLES

Implementation of the present disclosure is provided by way of the following examples. The examples serve to illustrate the technology without being limiting in nature.

### Example 1

### Inventive Compositions

| Inventive Compositions | | | A | B | C | D |
|---|---|---|---|---|---|---|
| (a) | Actives | CITRIC ACID | 1.2 | 1.2 | 1.2 | 1.6 |
| | | SODIUM CITRATE | 0.8 | 0.8 | 0.8 | 1.4 |
| Ratio of Citric Acid to Sodium Citrate | | | 1.5:1 | 1.5:1 | 1.5:1 | 1.1:1 |
| (b) | Cationic | CETRIMONIUM | 0.1 | 0.1 | 0.1 | |
| | Surfactnat | CHLORIDE | | | | |
| | | BEHENTRIMONIUM | 3.6 | 3.6 | 3.6 | 3.6 |
| | | CHLORIDE | | | | |
| | | DICETYLDIMONIUM | 1.1 | 1.1 | 1.1 | |
| | | CHLORIDE | | | | |
| | Amphoteric | COCO-BETAINE | 0.2 | 0.2 | 0.2 | |
| | Nonionic | PEG-100 STEARATE | ≤1 | ≤1 | ≤1 | ≤1 |
| | Surfactant | STEARETH-6 | | | | |
| | | TRIDECETH-3 | | | | |
| | | TRIDECETH-10 | | | | |
| | | POLYSORBATE 20 | | | | |
| (d) | Fatty | STEARYL ALCOHOL | | | | 1.8 |
| | Alcohol | CETEARYL | 12 | 12 | 12 | 7 |
| | | ALCOHOL | | | | |
| (e) | Fatty | ISOPROPYL | 0.5 | 0.5 | 0.5 | |
| | Compound | MYRISTATE | | | | |
| | | CETYL ESTERS | 1.5 | 1.5 | 1.5 | 1.5 |
| | | EUPHORBIA | 0.5 | 0.5 | 0.5 | |
| | | CERIFERA | | | | |
| | | (CANDELILLA) WAX | | | | |
| Ratio of (d) to (e) | | | 6 | 6 | 6 | 5.8 |
| (f) | Water- | GLYCERIN | 5 | 5 | 5 | 5 |
| | Solvent | | | | | |
| | Soluble | ISOPROPYL | 1.1 | 1.1 | 1.1 | 0.8 |
| | | ALCOHOL | | | | |
| (g) | Nonionic | HYDROXYPROPYL | 0.5 | 0.5 | 0.5 | 0.2 |
| | Thickening | GUAR | | | | |
| | Polymer | | | | | |
| (h) | Cationic | POLYQUATERNIUM- | | | | 0.3 |
| | Polymer | 10 | | | | |
| | Silicone | AMODIMETHICONE | 0.1 | 0.1 | 0.1 | |
| | | DIMETHICONE | 3.4 | 3.4 | 3.4 | |
| | | BIS-CETEARYL | 1.8 | 1.8 | 1.8 | |
| | | AMODIMETHICONE | | | | |
| (i) | Misc. | TRISODIUM HEDTA | ≤4 | ≤4 | ≤4 | ≤4 |
| | | SODIUM CHLORIDE | | | | |
| | | PHENOXYETHANOL | | | | |
| | | FRAGRANCE | | | | |
| | | ACETIC ACID, | | | | |
| | | DILAURYL | | | | |
| | | THIODIPROPIONATE | | | | |
| (c) | | WATER | 65 | 65 | 65 | 76 |

| | | | | | |
|---|---|---|---|---|---|
| Form | Emulsion | O/W | O/W | O/W | O/W |

| | | | | |
|---|---|---|---|---|
| pH | 3.5 | 3.5 | 3.5 | 3.8 |
| Viscosity | 39000* | 39000* | 39000* | 30000* |

*Viscosity was measured with T-bar E spindle using a Helipath stand mounted on Brookfield DV-II+ Pro, with 3 rotation speed of 20 RDM

### Example 2

### Fiber Integrity Testing

A study was carried out to evaluate hair fiber integrity of hair treated with Inventive Composition D from Example 1. The study used two different hair types: (1) European Medium Brown, 9% bleached hair tresses (3.0 g, 8″ length, 1″ wide); and (2) Curly multi-ethnic brown, 9% bleached hair tresses (3.0 g, 8″ length, 1″ wide). The hair swatches were subjected to one of three different treatment protocols.

| Treatment 1 | Treatment 2 | Treatment 3 |
|---|---|---|
| Shampoo | Inventive Composition D | Inventive Composition D, |
| (1 application) | (6 applications) | Shampoo, and |
| | | Conditioner |
| | | (6 applications) |

Treatment 1: 0.4 g of a standard shampoo was applied to hair swatches, massaged into the hair for 30 seconds, and then allowed to remain on the hair for 30 seconds. After 30 seconds, the shampoo was rinsed from the hair. The process was repeated six times (i.e., Treatment 1 was carried out six times).
Treatment 2: 0.4 g of Inventive Composition D was applied to hair swatches, massaged into the hair for 30 seconds, and then allowed to remain on the hair for 30 seconds. After 30 seconds, Inventive Composition was rinsed from the hair. The process was repeated six times (i.e., Treatment 2 was carried out six times).
Treatment 3: 0.4 g of Inventive Composition D was applied to hair swatches, massaged into the hair for 30 seconds, and then allowed to remain on the hair for 30 seconds. After 30 seconds, Inventive Composition D was rinsed from the hair. Next, 0.4 g of a standard shampoo was applied to the hair swatches, massaged into the hair for 30 seconds, and then allowed to remain on the hair for 30 seconds. After 30 seconds, the shampoo was rinsed from the hair. After rinsing the shampoo from the hair, 0.4 g of a standard conditioner was applied to the hair swatches, massaged into the hair for 30 seconds. After 30 seconds, the conditioner was rinsed from the hair. The process was repeated six times (i.e., Treatment 3 was carried out six times)

After being subjected to one of the three treatments described above, the hair swatches were blow dried for 2 minutes with a blow dryer set to medium heat. The hair fibers were periodically separated with fingers during the blow drying. After being dried, the hair swatches were stored at 60% relative humidity (60%) overnight.

After being stored overnight, hair fibers were randomly selected from each hair swatch for testing. The hair fibers were analyzed using a Dia-Stron Mini Tensile Tester (MTT) (MTT) to determine break stress, young's modulus, and break extension. The properties of hair are a direct consequence of its composite structure. Therefore, changes in break stress, Young's modulus, and break extension represent alteration in hair structure. A conventional approach for assessing the properties of hair is to generate stress-strain curves by performing constant rate extension experiments, from which information about the structure of the hair is obtained. FIG. 1 is an example of a stress-strain curve identifying the Young's modulus, plateau load, break force, and break extension.

Break stress represents the force/area needed to break the hair fiber. A higher break stress corresponds to a stronger fiber. For reference the typical dry state break stress for healthy, unbleached hair is about 0.022 gmf/sq micron.

Young's modulus is the slope of the initial portion of the stress-strain curve (see FIG. 1), which is adjusted for cross-sectional area. It is a measure of the hair “spring-like” properties. The Young's modulus region is sometimes referred to as the “linear region” or the “elastic region” of the curve.

Break extension is the percent extension of hair fibers at break point.

Box and whisker plots were generated using Statistica™, while JMP™ analytical software was used to calculate the statistics (student's t-test at 95% confidence level).

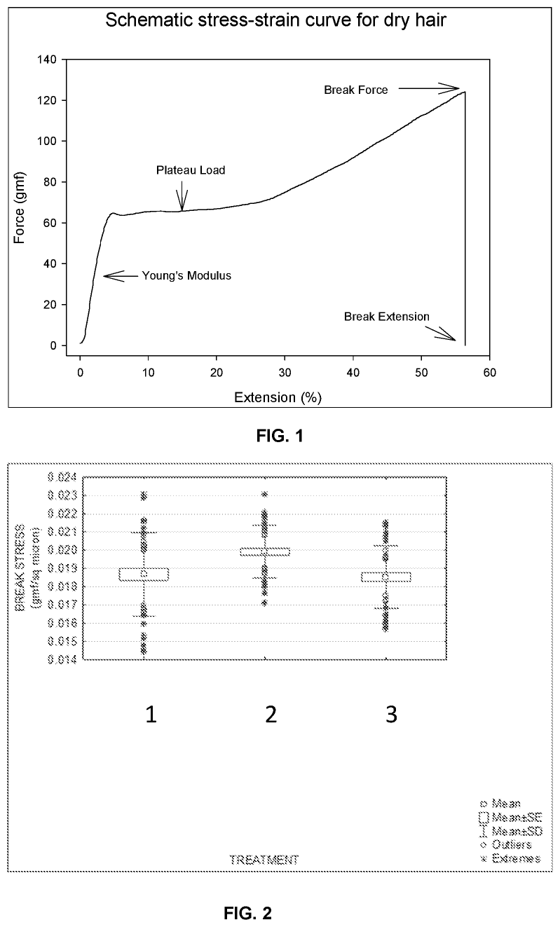

The break stress (gmf/sq micron) results for the European medium brown hair are provided in the table below and shown in FIG. 2.

Break Stress (gmf/sq micron)
European Medium Brown Hair

| Treatment | N | Mean | Std Dev | Std Err Mean | Differences* | |
|---|---|---|---|---|---|---|
| Treatment 2 | 50 | 0.0199 | 0.00146 | 0.000206 | A | |
| Treatment 1 | 50 | 0.0187 | 0.00227 | 0.000321 | | B |
| Treatment 3 | 50 | 0.0185 | 0.00171 | 0.000242 | | B |

*Levels not connected by same letter are significantly different.

The results show that the European medium brown hair treated according to Treatment 2 was statistically different from hair treated according to Treatments 1 and 3. The hair treated according to Treatment 2 exhibited statistically higher break stress results.

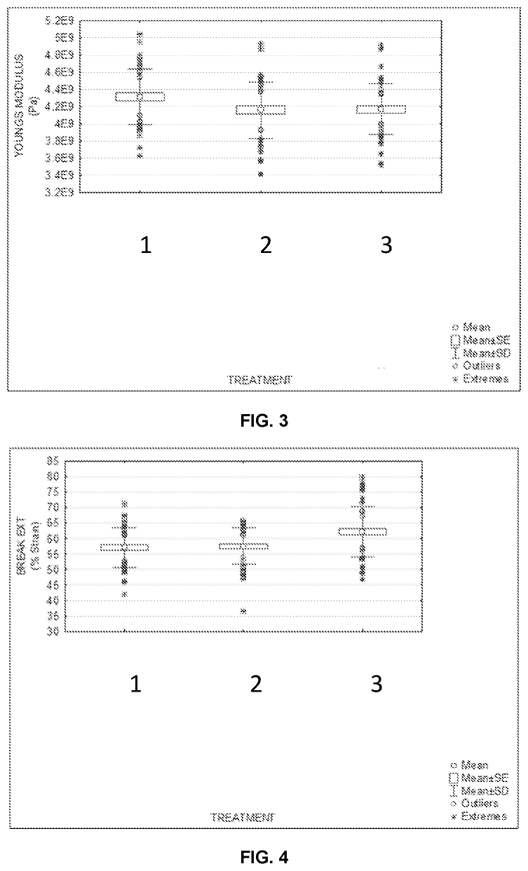

The Young's modulus results for the European medium brown hair are provided in the table below and shown in FIG. 3.

Young’s Modulus
European Medium Brown Hair

| Treatment | N | Mean | Std Dev | Std Err Mean | Differences* | |
|---|---|---|---|---|---|---|
| Treatment 1 | 50 | 4.31E+09 | 3.19E+08 | 4.52E+07 | A | |
| Treatment 2 | 50 | 4.16E+09 | 3.29E+08 | 4.66E+07 | | B |
| Treatment 3 | 50 | 4.17E+09 | 2.95E+08 | 4.17E+07 | | B |

*Levels not connected by same letter are significantly different.

The results show that the European medium brown hair treated according to Treatment 1 was statistically different from the hair treated according to Treatments 2 and 3.

The break extension results (% strain) for the European medium brown hair are provided in the table below and shown in FIG. 4.

Break Extension (% Strain)
European Medium Brown Hair

| Treatment | N | Mean | Std Dev | Std Err Mean | Differences* | |
|---|---|---|---|---|---|---|
| Treatment 3 | 50 | 62.1 | 8.04 | 1.137 | A | |
| Treatment 2 | 50 | 57.5 | 5.88 | 0.832 | | B |
| Treatment 1 | 50 | 57.1 | 6.41 | 0.906 | | B |

*Levels not connected by same let ter are significantly different

The results show that the Euro Medium Brown hair treated according to Treatment 3 is statistically different from hair treated according to Treatments 1 and 2. Hair treated according to Treatment 3 exhibited a statistically higher break extension.

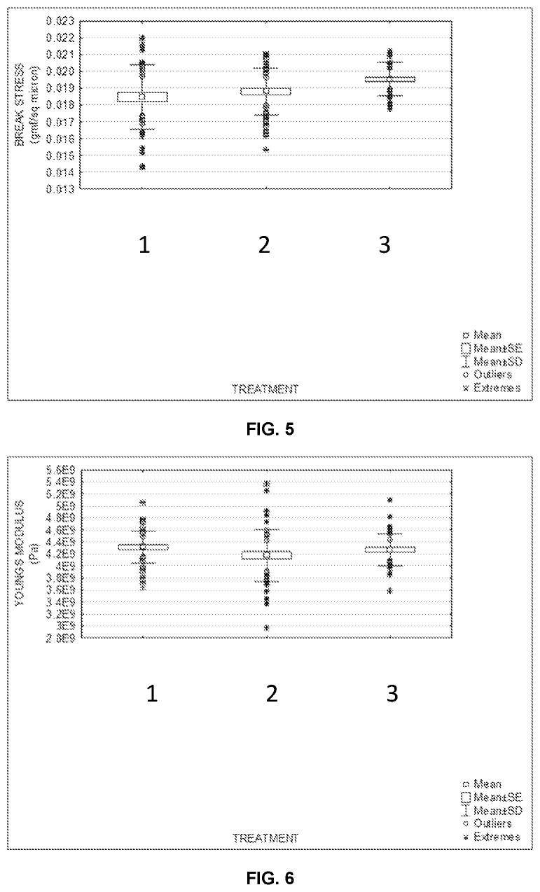

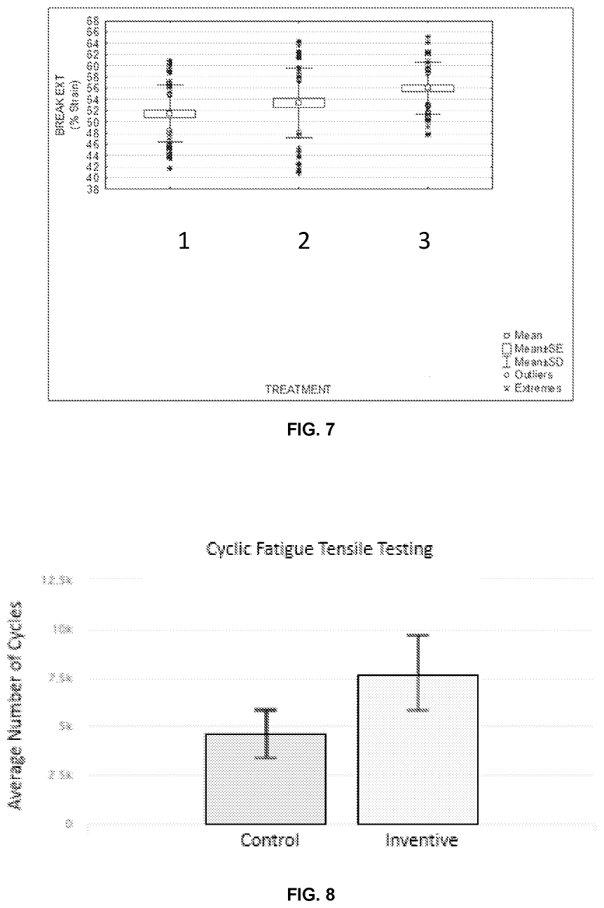

The break stress (gmf/sq micron) results for the curly multi-ethnic brown hair are provided in the table below and shown in FIG. 5.

Break Stress (gmf/sq micron)
Curly Multi-Ethnic Brown Hair

| Treatment | N | Mean | Std Dev | Std Err Mean | Differences* | |
|---|---|---|---|---|---|---|
| Treatment 3 | 50 | 0.0195 | 0.000996 | 0.000141 | A | |
| Treatment 2 | 50 | 0.0188 | 0.001386 | 0.000196 | | B |
| Treatment 1 | 50 | 0.0185 | 0.001930 | 0.000273 | | B |

*Levels not connected by same letter are significantly different.

The results show that the curly multi-ethnic hair treated according to Treatment 3 was statistically different from hair treated according to Treatments 1 and 2. Hair treated according to Treatment 3 showed a statistically higher break stress.

The Young's modulus for the curly multi-ethnic brown hair are provided in the table below and shown in FIG. 6.

Young’s Modulus
Curly Muti-Ethnic Brown Hair

| Treatment | N | Mean | Std Dev | Std Err Mean | Differences* | |
|---|---|---|---|---|---|---|
| Treatment 1 | 50 | 4.31E+09 | 2.65E+08 | 37492432 | A | |
| Treatment 3 | 50 | 4.27E+09 | 2.60E+08 | 36729080 | A | B |
| Treatment 2 | 50 | 4.18E+09 | 4.37E+08 | 61843278 | | B |

*Levels not connected by same letter are significantly different.

Break Extension (% Strain)
Curly Multi-Ethnic Brown Hair

| Treatment | N | Mean | Std Dev | Std Err Mean | Differences* | |
|---|---|---|---|---|---|---|
| Treatment 3 | 50 | 56.0 | 4.58 | 0.648 | A | |
| Treatment 2 | 50 | 53.4 | 6.18 | 0.874 | | B |
| Treatment 1 | 50 | 51.5 | 5.07 | 0.718 | | B |

*Levels not connected by same letter are significantly different

The results show that the curly multi-ethnic hair treated according to Treatment 3 is statistically different from hair treated according to Treatments 1 and 2. Hair treated according to Treatment 3 exhibited a statistically higher break extension.

### Example 3

### Cyclic Fatigue Testing

Inventive Composition D was subjected to cyclic fatigue testing. European highly bleached hair swatches were used for the testing (2.7 g, 27 cm). Two different hair tresses were used for each treatment group. 25 fibers were taken from each of the two tresses (total 50 fibers per treatment group) for testing. Each pair of hair tresses were treating with either a shampoo (Control) or with Inventive Composition D, a shampoo, and a conditioner (Inventive Treatment), as described below.
Control: 0.4 g of a standard shampoo was applied to hair swatches, massaged into the hair for 30 seconds, and then allowed to remain on the hair for 30 seconds. After 30 seconds, the shampoo was rinsed from the hair. The process was repeated 10 times.
Inventive
Treatment: 0.4 g of Inventive Composition D was applied to hair swatches, massaged into the hair for 30 seconds, and then allowed to remain on the hair for 5 minutes. After 5 minutes, Inventive Composition D was rinsed from the hair. Next, 0.4 g of a standard shampoo was applied to the hair swatches, massaged into the hair for 30 seconds, and then allowed to remain on the hair for 30 seconds. After 30 seconds, the shampoo was rinsed from the hair. After rinsing the shampoo from the hair, 0.4 g of a standard conditioner was applied to the hair swatches, massaged into the hair for 30 seconds. After 30 seconds, the conditioner was rinsed from the hair. The process was repeated 10 times.

After being subjected to one of the two treatments described above, the hair tresses were blow dried for 2 minutes with a blow dryer set to medium heat. The hair fibers were periodically separated from one another with fingers during the blow drying. After being dried, the hair swatches were stored at 60% relative humidity (60%) overnight.

After being stored overnight, hair fibers were randomly selected from each hair tress for testing. The hair fibers were subjected to cyclic fatigue tensile testing (CFTT) using a Dia-Stron Mini Tensile Tester (MTT). CFTT is a method for determining the durability of fibers. The CFTT was run under constant stress mode (using plateau stress obtained using MTT), and Kaplan-Meier statistical analysis was performed using

SPSS software. Hair swatches treated with the Inventive Treatment required more cycles to break than hair swatches treated with the Control Treatment. This illustrates that treatment with Inventive Composition D improves the durability of the hair and that multiple treatments further improve the durability of the hair.

The results are presented in the table below and graphically presented in FIG. 8.

Cyclic Fatigue Tensile Testing

| | Average cycles to |
|---|---|
| Treatment | break |
| Control | 4720 |
| Inventive Treatment | 7379 |

The results show a statistically significant improvement in strength (required more cycles to break) for hair treated with Inventive Composition D than the control.

### Definitions

The following definitions are in addition to those set forth throughout the instant disclosure.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the claimed invention.

The terms “a,” “an,” and “the” are understood to encompass the plural as well as the singular. Thus, the term “a mixture thereof” is interchangeable with “mixtures thereof.” Throughout the disclosure, the term “a mixture thereof” is used, following a list of elements as shown in the following example where letters A-F represent the elements: “one or more elements selected from the group consisting of A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements chosen from A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting.

The expression “one or more” means “at least one” and thus includes individual components as well as mixtures/combinations.

The term “plurality” means “more than one” or “two or more.”

Some of the various categories of components identified may overlap. In such cases where overlap may exist and the composition/product includes two overlapping components (or more than two overlapping components), an overlapping component does not represent more than one component. For example, a component such as a fatty ester may fall within a definition of a “fatty compound” and within a definition of an “ surfactant/emulsifier.” If a particular composition/product requires both a fatty compound component and an surfactant/emulsifier component, a single fatty ester can serve as only as a single fatty compound or a single surfactant/emulsifier (a single fatty ester cannot serve as both the fatty compound and the surfactant/emulsifier).

All percentages, parts and ratios herein are based upon weight, in particular, the total weight of the hair treatment compositions of the instant invention, unless otherwise indicated.

An “alkyl radical” is a linear or branched saturated hydrocarbon-based group, particularly C₁-C₈, more particularly C₁-C₆, preferably C₁-C₄ such as methyl, ethyl, isopropyl and tert-butyl;

An “alkoxy radical” is a alkyl-oxy wherein alkyl is as described herein before ;

An “alkenyl radical” is a linear or branched unsaturated hydrocarbon-based group, particularly C₂-C₈, more particularly C₂-C₆, preferably C₂-C₄ such as ethylenyl, propylenyl;

An “alkylene radical” is a linear or branched divalent saturated C₁-C₈, in particular C₁-C₆, preferably C₁-C₄ hydrocarbon-based group such as methylene, ethylene or propylene.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically points 1, 2, 3, 4 and 5, as well as sub-ranges such as 2-5, 3-5, 2-3, 2-4, 1-4, etc.; and points of 1, 2, 3, 4, and 5 includes ranges and sub-ranges of 1-5, 2-5, 3-5, 2-3, 2-4, 1-4, etc.

Other than in the operating examples, or where otherwise indicated, all numbers expressing quantities of ingredients and/or reaction conditions are understood to be modified by “about,” whether or not expressly stated. Additionally, all numbers are intended to represent exact figures as additional embodiments, whether or not modified by “about.” For example, “an amount of about 1%” includes an amount of exactly 1%. As a further example, “an amount of 1%” includes an amount of about 1%. The term “about” is generally understood to encompass a range of +/−10% from the stated number, and is intended to cover amounts of +/−1%, 2%, 3%, 4%, 5%, 6%, 7%, 8%, 9%, and 10%.

The term “surfactants” includes salts of the surfactants even if not explicitly stated. In other words, whenever the disclosure refers to a surfactant, it is intended that salts of the surfactant are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts with surfactants. However, additional cations such as ammonium ions, or alkanolammonium ions such as monoethanolammonium or triethanolammonium ions, may also form salts of surfactants.

The term “substantially free” or “essentially free” as used herein means the specific material may be present in small amounts that do not materially affect the basic and novel characteristics of the claimed invention. For instance, there may be less than 2% by weight of a specific material added to a composition, based on the total weight of the compositions (provided that an amount of less than 2% by weight does not materially affect the basic and novel characteristics of the claimed invention). Similarly, the compositions may include less than 2 wt %, less than 1.5 wt %, less than 1 wt %, less than 0.5 wt %, less than 0.1 wt %, less than 0.05 wt %, or less than 0.01 wt %, or none of the specified material. The term “substantially free” or “essentially free” as used herein may also mean that the specific material is not added to the composition but may still be present in a raw material that is included in the composition.

Furthermore, all components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair treatment composition comprising:
(a) about 0.25 to about 10 wt. % of citric acid, one or more salts of citric acid, or a combination thereof,
wherein if a combination of citric acid and salts of citric acid is present, the weight ratio of citric acid to salts of citric acid added to form the combination is from 1:10 to about 10:1,
alternatively, however, the hair treatment composition comprises about 1 to about 10 wt. % of a combination of citric acid and one or more salts of citric acid,
wherein the weight ratio of citric acid to salts of citric acid added to form the combination is from 1:10 to about 10:1;
(b) one or more surfactants; and
(c) water;
wherein the pH of the composition is from about 3 to about 10; and
weight percentages are based on the total weight of the composition.

2. The hair treatment composition of claim 1, wherein at least one of the one or more surfactants of (b) is a cationic surfactant.

3. The hair treatment composition of claim 2, wherein at least one of the cationic surfactants is selected from cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, brassicamidopropyldimethylamine, lauramidopropyl dimethylamine, myristamidopropyl dimethylamine, dilinoleamidopropyl dimethylamine, palmitamidopropyl dimethylamine, and mixtures thereof.

4. The hair treatment composition of claim 1, wherein at least one of the one or more surfactants of (b) is a nonionic surfactant, an amphoteric surfactant, or a combination thereof; and the composition further comprises:
(d) one or more fatty alcohols; and
(e) one or more fatty compounds other than fatty alcohols;
wherein the composition is in the form of an oil in water emulsion.

5. The hair treatment composition of claim 4 comprising:
(d) about 1 to about 15 wt. % of the one or more fatty alcohols; and
(e) about 1 to about 10 wt. %, of the one or more fatty compounds other than fatty alcohols;
wherein the weight ratio of (d) to (e) is from about 2:1 to about 15:1.

6. The hair treatment composition of claim 5, wherein the one or more fatty compounds other than fatty alcohols are chosen from oils, waxes, alkanes (paraffins), fatty acids, fatty esters, triglyceride compounds, lanolin, derivatives thereof, and mixtures thereof.

7. The hair treatment composition of claim 1, further comprising:
(f) one or more water-soluble solvents.

8. The hair treatment composition of claim 7, wherein the one or more water-soluble solvents are chosen from glycerin, C₁₋₆ mono-alcohols, polyols (polyhydric alcohols), glycols, and a mixture thereof.

9. The hair treatment composition claim 1, further comprising:
(g) one or more nonionic thickening polymers.

10. The hair treatment composition of claim 9, wherein the one or more nonionic thickening polymers are chosen from guar gum, guar derivatives, cellulose gum, cellulose derivatives, starch, starch derivatives, polysaccharides, polysaccharide derivatives, homopolymers and copolymers of ethylene oxide having a molar mass equal to or greater than 10,000 g/mol, polyvinyl alcohols, homopolymers and copolymers of vinylpyrrolidone, homopolymers and copolymers of vinylcaprolactam, homopolymers and copolymers of polyvinyl methyl ether, and mixtures thereof.

11. The hair treatment composition claim 1, further comprising:
(h) one or more conditioning agents chosen from cationic polymers, silicones, and a mixture thereof.

12. The hair treatment composition of claim 11 comprising one or more cationic polymers chosen from cationic cellulose derivatives (e.g., polyquaternium-10), quaternized hydroxyethyl cellulose, cationic starch derivatives, cationic guar gum derivatives, cationic proteins and cationic protein hydrolysates, quaternary diammonium polymers, copolymers of acrylamide and dimethyldiallyammonium chloride, polyquaterniums (e.g., polyquaternium-10), and a mixture thereof.

13. The hair treatment composition of claim 11 comprising one or more silicones chosen from dimethicone, dimethiconol, cyclomethicone, polysilicone-11, phenyl trimethicone, amodimethicone, bis-cetearyl amodimethicone, and a mixture thereof.

14. A hair treatment composition comprising:
(a) about 0.25 to about 10 wt. % of citric acid, one or more salts of citric acid, or a combination thereof,
wherein if a combination of citric acid and salts of citric acid is present, the weight ratio of citric acid to salts of citric acid added to form the combination is from 1:10 to about 10:1,
alternatively, however, the hair treatment composition comprises about 1 to about 10 wt. % of a combination of citric acid and one or more salts of citric acid;
wherein the weight ratio of citric acid to salts of citric acid added to form the combination is from 1:10 to 10:1;
(b) one or more cationic surfactants and one or more surfactants chosen from nonionic surfactants, amphoteric surfactants, and a mixture thereof;
(c) water;
(d) one or more fatty alcohols;
(e) one or more fatty compounds other than fatty alcohols;
wherein the weight ratio of (d) to (e) is from about 2:1 to about 15:1;
(f) one or more water-soluble solvents;
(g) one or more nonionic thickening polymers;
(h) optionally, one or more conditioning agents chosen from cationic polymers, silicones, and mixtures thereof;
wherein the pH of the composition is from about 3 to about 10; and
weight percentages are based on the total weight of the composition.

15. The hair treatment composition of claim 14 in the form of an oil in water emulsion.

16. The hair treatment composition of claim 14 comprising:
(a) about 0.25 to about 10 wt. % of citric acid, one or more salts of citric acid, or a combination thereof,
wherein if a combination of citric acid and salts of citric acid is present, the weight ratio of citric acid to salts of citric acid added to form the combination is from 1:10 to about 10:1,
alternatively, however, the hair treatment composition comprises about 1 to about 10 wt. % of a combination of citric acid and one or more salts of citric acid;
wherein the weight ratio of citric acid to salts of citric acid added to form the combination is from 1:10 to 10:1;
(b) about 1 to about 10 wt. % of one or more cationic surfactants;
(c) about 50 to about 80 wt. % of water;
(d) about 5 to about 15 wt. % of one or more fatty alcohols;
(e) about 1 to about 10 wt. % of one or more fatty compounds other than fatty alcohols;
(f) about 0.5 to about 10 wt. % of one or more water-soluble solvents;
(g) about 0.1 to about 5 wt. % of one or more nonionic thickening polymers;
(h) about 0.1 to about 10 wt. % of one or more conditioning agents chosen from cationic polymers, silicones, and mixtures thereof;
(i) about 0.01 to about 5 wt. % of one or more miscellaneous ingredients;
wherein the composition is in the form of an oil in water emulsion and the pH of the composition is from about 3 to about 10.

17. A method for treating hair comprising applying a hair treatment composition to the hair, the hair treatment composition comprising:
(a) about 0.25 to about 10 wt. % of citric acid, one or more salts of citric acid, or a combination thereof; or about 1 to about 10 wt. % of citric acid and at least one salt of citric acid;
(b) one or more surfactants; and
(c) water;
wherein the pH of the composition is from about 3 to about 10; and
weight percentages are based on the total weight of the composition.

18. The method of claim 17 further comprising rinsing the hair treatment from the hair after the application has been applied to the hair for a period of about 30 seconds to about 5 minutes.

19. The method of claim 17, wherein the composition is a leave-on hair treatment composition and the compositions remains on the hair after application without rinsing the hair and the hair is styled.

20. The method of claim 17, wherein the method strengthens the hair fibers.
